(19) United States
(12) Patent Application Publication
(10) Pub. No.: US 2018/0318024 A1
(43) Pub. Date: Nov. 8, 2018

(54) MEDICAL INSTRUMENT

(71) Applicant: OLYMPUS CORPORATION, Tokyo (JP)

(72) Inventor: Toshihiro YOSHII, Tokyo (JP)

(73) Assignee: OLYMPUS CORPORATION, Tokyo (JP)

(21) Appl. No.: 16/039,552

(22) Filed: Jul. 19, 2018

Related U.S. Application Data
(63) Continuation of application No. PCT/JP2016/051829, filed on Jan. 22, 2016.

(51) Int. Cl. A61B 34/37; A61B 34/00; F16H 19/00; B25J 3/04; B25J 17/00; B25J 11/00

(52) U.S. Cl. CPC A61B 34/37; A61B 34/71; F16H 19/001; A61B 2034/301; B25J 17/00; B25J 11/008; B25J 3/04

(57) ABSTRACT

A medical instrument includes a first joint. The first joint has a first axis, a second axis, a third axis, a first connection member which has a first rotation-transmitting portion, a second connection member which has a second rotation-transmitting portion, a third connection member, and a third rotation-transmitting portion. The first rotation-transmitting portion and the second rotation-transmitting portion are connected to each other so as to pivot by interlocking with each other. The third connection member has a fourth rotation-transmitting portion which is provided in an end portion of the third connection member and which is connected to the third rotation-transmitting portion so as to pivot about the first axis or the second axis by interlocking with the third rotation-transmitting portion.

This application is a continuation application based on a PCT Patent Application No. PCT/JP2016/051829, filed on Jan. 22, 2016. The content of the PCT Application is incorporated herein by reference.

### BACKGROUND OF THE INVENTION

### Field of the Invention

The present invention relates to a medical instrument.

### Description of Related Art

In the related art, as a medical manipulator, a master-slave-type medical manipulator including a master manipulator that is operated by an operator, and a slave manipulator that makes a movement based on a signal emitted from the master manipulator is known. A medical instrument for treating a treatment target site through a remote operation is attached to such a medical manipulator. The medical instrument includes an end effector that is provided in a distal end, and a joint that changes the direction of the end effector. U.S. Pat. No. 5,784,542 discloses a structure of a double joint articulation which can be applied to the joint of this medical instrument.

Japanese Unexamined Patent Application, First Publication No. 2006-116194 discloses a medical instrument having a double joint articulation. The medical instrument includes a gripping portion (end effector) that grips a suture thread or a needle, a distal end portion that is positioned on a proximal end side of the gripping portion, an intermediate portion that forms a second articulation together with the distal end portion, and a root portion that forms a first articulation together with the intermediate portion. The gripping portion has a pair of blades to which a drive wire is attached via a blade pulley. The distal end portion has a semicircular gear portion which is provided on the proximal end side and is penetrated by a first axis. The root portion has a semicircular gear portion which is provided on the distal end side and is penetrated by a second axis. The intermediate portion is formed such that the distal end portion and the root portion are respectively pivotable about the axes of the first and the second axes. In the intermediate portion, the gear portion of the distal end portion and the gear portion of the root portion engage with each other. The intermediate portion has an intermediate blade which is arranged to be adjacent to the gear portions and is penetrated by each of the first and the second axes. A wire is fixed to the intermediate blade. When the wire is pulled to the proximal end side, the intermediate blade pivots about the second axis, and while the gear portion of the distal end portion and the gear portion of the root portion make an engaging movement, the directions of the distal end portion and the gripping portion with respect to the root portion change.

In addition, the intermediate portion has a pulley rotatable around the first axis and a pulley rotatable around the second axis which are arranged such that the intermediate blade and the gear portion are interposed therebetween. The drive wire for operating the pair of blades is hung to be introduced from the pulley of the first axis to the pulley of the second axis. In a double joint articulation having a configuration as described above, even in a case where the distal end portion is bent with respect to the root portion, a path length of the drive wire does not change. Therefore, the pair of blades can be operated without interfering with other articulations.

In a medical instrument disclosed in Japanese Unexamined Patent Application, First Publication No. 2006-116194, in order to apply a significant force to a pair of blades of a gripping portion, there is a need to apply a significant tensile force to a drive wire. In this case, since the drive wire is hung on pulleys of an intermediate portion, a force corresponding to a tensile force of the drive wire is also applied, via the pulley, to a first axis and a second axis respectively supporting the pulleys. In a case where the tensile force of the drive wire is significant, there is a possibility that the first axis and the second axis will be relatively tilted due to the tensile force. In this case, since teeth of a gear portion of a distal end portion and teeth of a gear portion of a root portion engage with each other in a tilted state, a frictional force between the gear portions increases. As a result, controllability or operability of the blades deteriorates.

There is a possibility that this problem may also occur in a medical instrument having a plurality of double joint articulations. Generally, a double joint articulation has a reduction ratio smaller than that of an ordinary articulation. Therefore, in order to bend a double joint articulation, there is a need to apply a significant tensile force to an operation wire for operating the double joint articulation. The operation wire of the double joint articulation provided on a distal end side is hung on the pulley arranged in the double joint articulation provided on a proximal end side of the articulation. Therefore, in a case where a significant tensile force is applied to the operation wire of the double joint articulation on the distal end side, there is a possibility that the above-described problem will occur in the double joint articulation on the proximal end side.

In addition to a case where the drive wire of the gripping portion is operated, or a case where the operation wire of the double joint articulation is operated as described above, there is a possibility that the above-described problem will also occur even when a certain external force is applied to the double joint articulation and the axes are tilted. That is, it is possible to say that the above-described problem will occur in a case where an external disturbance is applied to the double joint articulation.

### SUMMARY OF THE INVENTION

An object of the present invention is to provide a medical instrument having a double joint articulation resistant to an external disturbance.

According to a first aspect of the present invention, a medical instrument includes a first joint which is provided between a first arm that is arranged in a distal end side of the medical instrument and a second arm that is arranged in a proximal end side of the medical instrument. The first joint has a first axis which is a pivot center of the first joint, a second axis which is provided to be separated from the first axis and is arranged to be parallel to the first axis, a third axis which is provided to be separated from both of the first axis and the second axis and is arranged to be parallel to the first axis and the second axis, a first connection member which has a first rotation-transmitting portion pivotable about the first axis and is fixed to the first arm, a second connection member which has a second rotation-transmitting portion pivotable about the second axis and is fixed to the second arm, a third connection member which is pivotable about the first axis and is pivotable about the second axis, and a third rotation-transmitting portion which is pivotable about the third axis. The first rotation-transmitting portion and the second rotation-transmitting portion are connected to each other to pivot by interlocking with each other. The third connection member has a fourth rotation-transmitting portion which is provided in an end portion of the third connection member and which is connected to the third rotation-transmitting portion to pivot about the first axis or the second axis by interlocking with the third rotation-transmitting portion.

According to a second aspect of the present invention, in the medical instrument according to the first aspect, the third axis may be arranged on a proximal end side of the second axis and a position of the third axis may be fixed with respect to the second axis.

The fourth rotation-transmitting portion may be connected to the third rotation-transmitting portion to pivot about the second axis by interlocking with the third rotation-transmitting portion.

According a third aspect of the present invention, in the medical instrument according to the second aspect, the first rotation-transmitting portion may have a sector-shaped first site about the first axis and may have a first gear portion in which teeth are formed on an arc of the first site. The second rotation-transmitting portion may have a sector-shaped second site about the second axis and may have a second gear portion in which teeth are formed on an arc of the second site. The first gear portion and the second gear portion may engage with each other. The fourth rotation-transmitting portion may have a sector-shaped third site about the second axis and may have a third gear portion in which teeth are formed on an arc of the third site. The third rotation-transmitting portion may have a fourth gear portion which is formed into a columnar shape about the third axis and in which teeth are formed on an outer circumferential surface around the third axis. The third gear portion and the fourth gear portion may engage with each other.

According to a fourth aspect of the present invention, in the medical instrument according to the third aspect, a pitch circle diameter of the fourth gear portion may be smaller than a pitch circle diameter of the third gear portion.

According to a fifth aspect of the present invention, in the medical instrument according to the third or fourth aspect, the first joint may further have a rotor which is pivotable about the third axis, and an operation member which is wound around the rotor. The rotor may be connected to the fourth gear portion to integrally pivot with the fourth gear portion.

According to a sixth aspect of the present invention, in the medical instrument according to the fifth aspect, an outer diameter of the rotor may be larger than the pitch circle diameter of the fourth gear portion.

According to a seventh aspect of the present invention, in the medical instrument according to any one of the second to sixth aspects, the first rotation-transmitting portion may be divided into a first part and a second part in a direction along the first axis. The second rotation-transmitting portion may be divided into a third part and a fourth part in a direction along the second axis. The third connection member may be arranged between the first part and the second part of the first rotation-transmitting portion and may be arranged between the third part and the fourth part of the second rotation-transmitting portion.

According to an eighth aspect of the present invention, in the medical instrument according to any one of the second to seventh aspects, the second arm may have a third arm which is connected to the first joint, a second joint which is connected to a proximal end side of the third arm, and a fourth arm which connects a proximal end side of the second joint and the main body to each other. The second joint may have a fourth axis; a fifth axis which is provided to be separated from the fourth axis and is arranged to be parallel to the fourth axis; a sixth axis which is provided to be separated from both of the fourth axis and the fifth axis, which is arranged to be parallel to the fourth axis and the fifth axis on a distal end side of the fourth axis, and of which a position is fixed with respect to the fourth axis; a fourth connection member which has a fifth rotation-transmitting portion pivotable about the fourth axis and is fixed to the third arm; a fifth connection member which has a sixth rotation-transmitting portion pivotable about the fifth axis and is fixed to the fourth arm; a sixth connection member which is pivotable about the fourth axis and is pivotable about the fifth axis; and a seventh rotation-transmitting portion which is pivotable about the sixth axis. The fifth rotation-transmitting portion and the sixth rotation-transmitting portion may be connected to each other to pivot by interlocking each other. The sixth connection member may have an eighth rotation-transmitting portion which is provided in an end portion of the sixth connection member and which is connected to the seventh rotation-transmitting portion to pivot about the fourth axis by interlocking with the seventh rotation-transmitting portion.

According to a ninth aspect of the present invention, in the medical instrument according to the eighth aspect, the fifth rotation-transmitting portion may have a sector-shaped fourth site about the fourth axis and may have a fifth gear portion in which teeth are formed on an arc of the fourth site. The sixth rotation-transmitting portion may have a sector-shaped fifth site about the fifth axis and may have a sixth gear portion in which teeth are formed on an arc of the fifth site. The fifth gear portion and the sixth gear portion may engage with each other. The eighth rotation-transmitting portion may have a sector-shaped sixth site about the fourth axis and may have a seventh gear portion in which teeth are formed on an arc of the sixth site. The seventh rotation-transmitting portion may have an eighth gear portion which is formed into a columnar shape about the sixth axis and in which teeth are formed on an outer circumferential surface around the sixth axis. The seventh gear portion and the eighth gear portion may engage with each other.

According to a tenth aspect of the present invention, in the medical instrument according to the ninth aspect, a pitch circle diameter of the eighth gear portion may be smaller than a pitch circle diameter of the seventh gear portion.

According to an eleventh aspect of the present invention, in the medical instrument according to the first aspect, the third axis may be arranged on a distal end side of the first axis and a position of the third axis may be fixed with respect to the first axis. The fourth rotation-transmitting portion may be connected to the third rotation-transmitting portion to pivot about the first axis by interlocking with the third rotation-transmitting portion.

According to a twelfth aspect of the present invention, in the medical instrument according to the eleventh aspect, the first rotation-transmitting portion may have a sector-shaped first site about the first axis and may have a first gear portion in which teeth are formed on an arc of the first site. The second rotation-transmitting portion may have a sector-shaped second site about the second axis and may have a second gear portion in which teeth are formed on an arc of the second site. The first gear portion and the second gear portion may engage with each other. The fourth rotation-transmitting portion may have a sector-shaped third site about the first axis and may have a third gear portion in which teeth are formed on an arc of the third site. The third rotation-transmitting portion may have a fourth gear portion which is formed into a columnar shape about the third axis and in which teeth are formed on an outer circumferential surface around the third axis. The third gear portion and the fourth gear portion may engage with each other.

According to a thirteenth aspect of the present invention, in the medical instrument according to the twelfth aspect, a pitch circle diameter of the fourth gear portion may be smaller than a pitch circle diameter of the third gear portion.

According to a fourteenth aspect of the present invention, in the medical instrument according to the twelfth or thirteenth aspect, the first joint may further have a rotor which is pivotable about the third axis, and an operation member which is wound around the rotor. The rotor may be connected to the fourth gear portion to integrally pivot with the fourth gear portion.

According to a fifteenth aspect of the present invention, in the medical instrument according to the fourteenth aspect, an outer diameter of the rotor may be larger than the pitch circle diameter of the fourth gear portion.

According to a sixteenth aspect of the present invention, in the medical instrument according to the eleventh to fifteenth aspects, the first rotation-transmitting portion may be divided into a first part and a second part in a direction along the first axis. The second rotation-transmitting portion may be divided into a third part and a fourth part in a direction along the second axis. The third connection member may be arranged between the first part and the second part of the first rotation-transmitting portion and may be arranged between the third part and the fourth part of the second rotation-transmitting portion.

According to a seventeenth aspect of the present invention, a medical instrument includes: a first rotation means for rotating a first connection member that is fixed to an arm; a second rotation means for rotating a first connection member with respect to a second connection member, the second rotation means being provided in the second connection member that interlocks with the first connection member; a third rotation means for rotating in the second connection member, the third rotation means being arranged opposed to the first rotation means with respect to the second rotation means; a fourth rotation means for rotating around the first rotation means, the fourth rotation means being provided to a third connection member that is connected to the first connection member; and an operation means for operating the third rotation means. The third connection member that interlocks with the third rotation means rotates with respect to the first rotation means by the fourth rotation means by operating the third rotation means by the operation means, and the first connection member is bent with respect to the second connection member by rotating the first connection member around the first rotation means by the second rotation means.

According to an eighteenth aspect of the present invention, in medical instrument according to the seventeenth aspect, a rotation direction of the first connection member may be changed with respect to the second connection member by changing the rotation direction with respect to the third rotation means by the operation means.

### BRIEF DESCRIPTION OF THE DRAWINGS

FIG. 1 is a general diagram of a medical manipulator used together with a medical instrument according to an embodiment of the present invention.

FIG. 2 is a perspective view illustrating a distal end side of the medical instrument.

FIG. 3 is a plan view illustrating a configuration of an end effector of the medical instrument.

FIG. 4 is a cross-sectional view taken along line A-A in FIG. 3.

FIG. 5 is a part of a cross-sectional view illustrating a configuration of a distal end joint of the medical instrument.

FIG. 6 is an exploded perspective view of the distal end joint.

FIG. 7 is a perspective view illustrating a configuration of an intermediate connection member and a rotation-transmitting portion of the distal end joint.

FIG. 8 is a diagram illustrating a state where the distal end joint is straight.

FIG. 9 is a diagram illustrating a state where the distal end joint is bent.

FIG. 10 is a part of a cross-sectional view illustrating a configuration of an intermediate joint of the medical instrument.

FIG. 11 is an exploded perspective view of the intermediate joint.

FIG. 12 is a perspective view illustrating a configuration of an intermediate connection member and a rotation-transmitting portion of the intermediate joint.

FIG. 13 is a diagram illustrating a state where the intermediate joint is straight.

FIG. 14 is a diagram illustrating a state where the intermediate joint is bent.

### DETAILED DESCRIPTION OF THE INVENTION

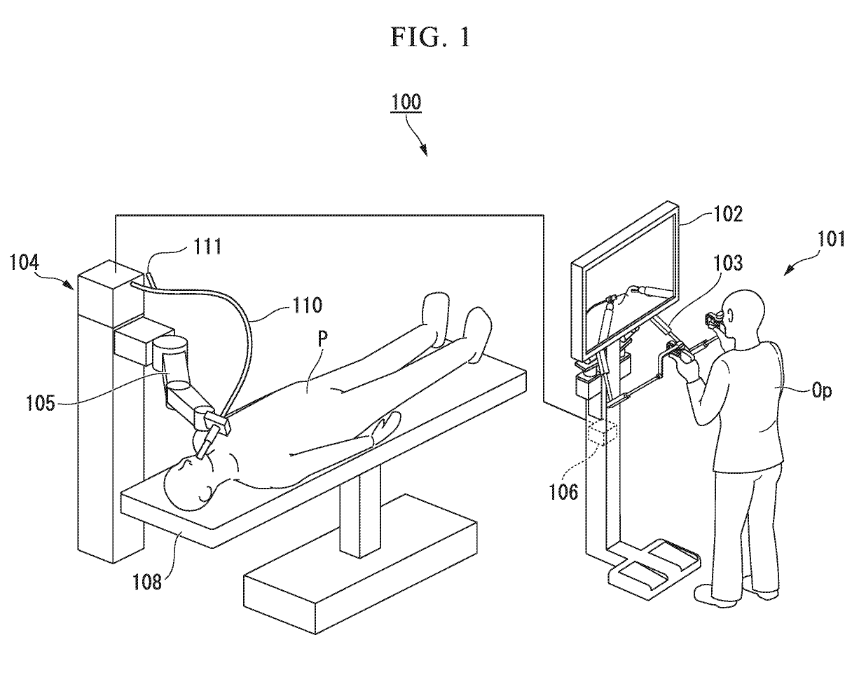

Hereinafter, an embodiment of the present invention will be described with reference to FIG. 1 to FIG. 15.

FIG. 1 is a general diagram illustrating an example of a medical manipulator 100 used together with a medical instrument according to the present embodiment. The medical manipulator 100 has a master-slave-type remote operation system. The medical manipulator 100 includes a master manipulator 101, a slave manipulator 104, and a control device 106.

The master manipulator 101 functions as a master unit transmitting a movement of an operation of an operator Op to the slave manipulator 104. The master manipulator 101 includes a display unit 102 such as a liquid crystal display, and a master arm 103 which the operator Op grips to perform an operation. The master arm 103 has a known configuration allowing multi-axis movement. An operation performed with respect to the master arm 103 is input to the control device 106.

The control device 106 includes a master control unit (not shown) receiving an input from the master manipulator 101, and a slave control unit (not shown) outputting a drive signal to the slave manipulator 104. The master control unit generates an operation command for making a movement of the slave manipulator 104 based on an input from the master manipulator 101 and outputs the operation command to the slave control unit. The slave control unit generates a drive signal for driving the slave manipulator 104 based on an operation command output from the master control unit and outputs the drive signal to the slave manipulator 104.

The slave manipulator 104 is installed beside a surgical bed 108 on which a patient P is placed. The slave manipulator 104 includes a slave arm 105 making a movement in response to a drive signal output from the slave control unit. The slave arm 105 is configured to have a plurality of articulations having multiple degrees of freedom and is able to make a multi-axis movement. Each of the articulations having multiple degrees of freedom is individually driven by a power unit (not shown). As the power unit, for example, a motor (servo motor) having a servo mechanism provided with an increment encoder or a speed reducer can be used.

A flexible endoscope 110 is attached to the slave manipulator 104. The endoscope 110 is supported by a distal end portion of the slave arm 105 and is inserted into the body of the patient P. The endoscope 110 is provided with a channel (not shown) through which the medical instrument can be inserted. The medical instrument according to the present embodiment is used by being inserted through the channel from an insertion port 111 provided on a proximal end side of the endoscope 110. In addition, the endoscope 110 is provided with observation means for acquiring a video image inside the body. The display unit 102 displays a video image acquired by the observation means.

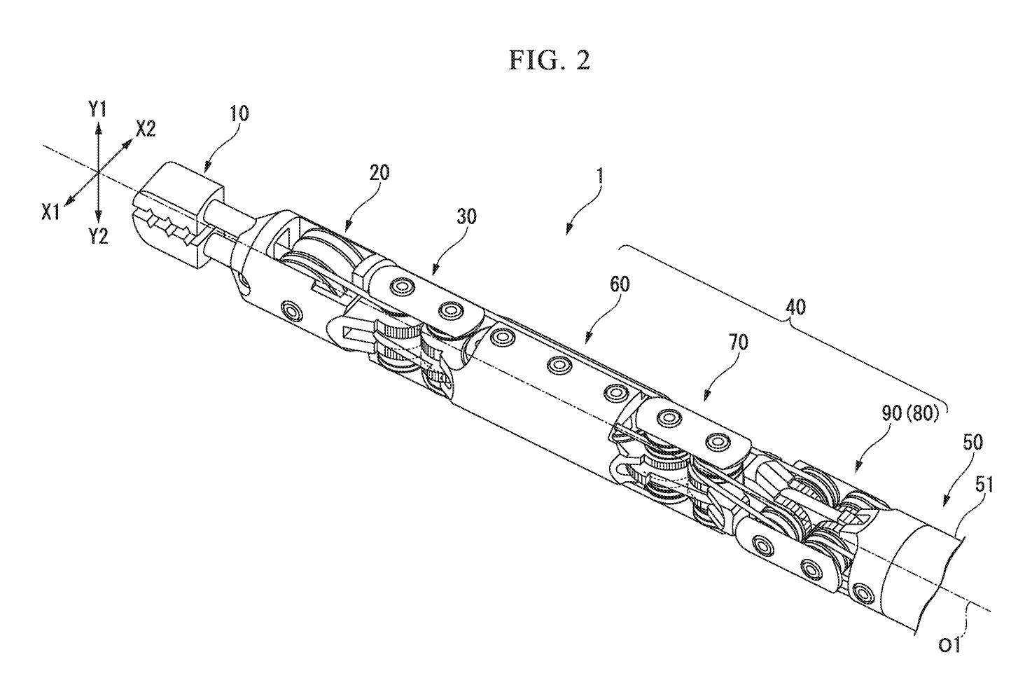

Next, a medical instrument 1 according to the present embodiment will be described. FIG. 2 is a perspective view illustrating a distal end side of the medical instrument 1. The medical instrument 1 is formed in a longitudinal shape along a longitudinal axis O1.

In the following description, a side close to a treatment target site in the longitudinal axis O1 of the medical instrument 1 when in use is referred to as a “distal end side”, and the opposite side of the distal end side is referred to as a “proximal end side”. In addition, in members, an end portion on the distal end side is referred to as a “distal end portion”, and an end portion on the proximal end side is referred to as a “proximal end portion”. In addition, two directions orthogonal to the longitudinal axis O1 and orthogonal to each other are referred to as a direction X1 and a direction Y1. In addition, a direction opposite to the direction X1 is referred to as a direction X2, and a direction opposite to the direction Y1 is referred to as a direction Y2.

As shown in FIG. 2, the medical instrument 1 includes an end effector 10, a distal end arm (first arm) 20 connected to the end effector 10, a distal end joint (first joint) 30 connected to the proximal end side of the distal end arm 20, an intermediate arm (second arm) 40 connected to the proximal end side of the distal end joint 30, and a main body 50 connected to the proximal end side of the intermediate arm 40. In addition, in the present embodiment, the intermediate arm 40 has an arm (third arm) 60 connected to the distal end joint 30, an intermediate joint (second joint) 70 connected to the proximal end side of the arm 60, and an arm (fourth arm) 80 connecting the proximal end side of the intermediate joint 70 and the main body 50 to each other. The arm 80 has a proximal end joint 90. In the medical instrument 1, the end effector 10, the distal end arm 20, the distal end joint 30, the arm 60, the intermediate joint 70, the proximal end joint 90 (arm 80), and the main body 50 are arranged along the longitudinal axis O1 in this order from the distal end side to the proximal end side. In addition, each of the distal end joint 30, the intermediate joint 70, and the proximal end joint 90 independently makes a bending movement.

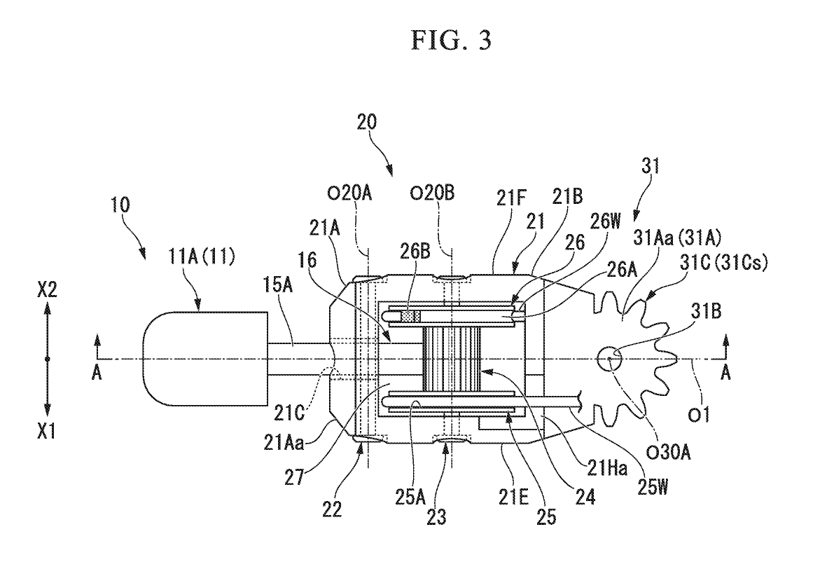

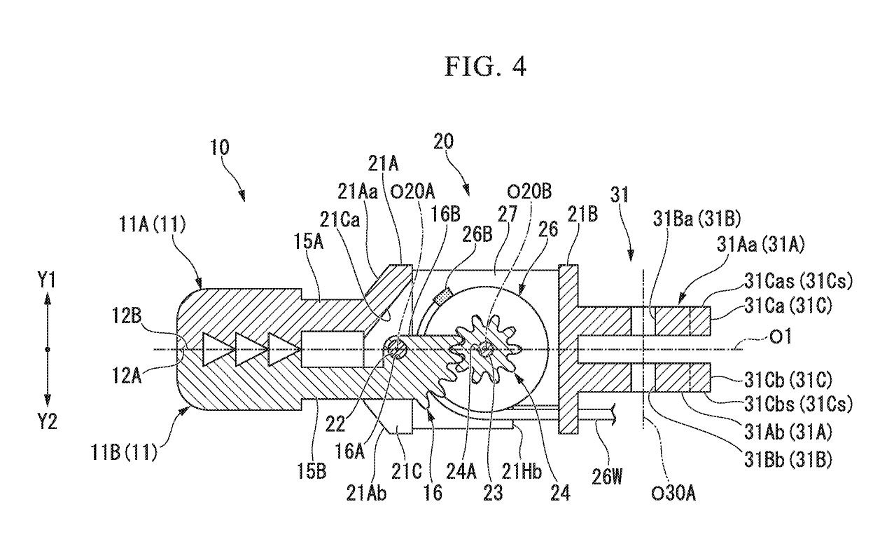

FIG. 3 is a plan view illustrating a configuration of the end effector 10 and the distal end arm 20. FIG. 4 is a cross-sectional view taken along line A-A in FIG. 3. The present embodiment illustrates an example in which the medical instrument 1 is gripping forceps. The end effector 10 has a pair of gripping pieces 11 which is configured to have a first gripping piece 11A and a second gripping piece 11B and is able to be opened and closed. A plurality of projection portions and recess portions are alternately formed on a surface 12A of the first gripping piece 11A facing the second gripping piece 11B. Similarly, a plurality of projection portions and recess portions are formed on a surface 12B of the second gripping piece 11B facing the first gripping piece 11A to face the plurality of projection portions and recess portions of the surface 12A of the first gripping piece 11A.

A connection member 15A is provided on the proximal end side of the first gripping piece 11A. The first gripping piece 11A is fixed to the distal end arm 20 via the connection member 15A. A connection member 15B is provided on the proximal end side of the second gripping piece 11B. A gear portion 16 is provided in the proximal end portion of the connection member 15B. The gear portion 16 is pivotably attached to a pivot axis 22 which extends in the direction X1 inside the distal end arm 20 and engages with the distal end arm 20. A circular hole 16A penetrating the gear portion 16 in the direction X1 about an axis O20A orthogonal to the longitudinal axis O1 and parallel to the direction X1 is formed in the gear portion 16. The pivot axis 22 formed into a columnar shape extending in the direction X1 and having the axis O20A as its center axis is inserted through the circular hole 16A. Both ends of the pivot axis 22 are supported by a distal end portion 21A of a main body portion 21. The pivot axis 22 is relatively and pivotably supported by the gear portion 16 via the circular hole 16A about the axis O20A. That is, the gear portion 16 and the second gripping piece 11B connected to the gear portion 16 are pivotable about the axis O20A with respect to the pivot axis 22. When the second gripping piece 11B pivots with respect to the first gripping piece 11A, the pair of gripping pieces 11 is opened and closed in the direction Y1 and the direction Y2. In addition, the gear portion 16 has a sector-shaped site about the axis O20A on the proximal end side of the circular hole 16A, and teeth are formed on an arc of the sector-shaped site. The gear portion 16 is configured to engage with a gear portion 24 provided in the distal end arm 20.

The distal end arm 20 has the main body portion 21, the gear portion 24, a pulley 25, and a pulley 26. The main body portion 21 has a substantially columnar external shape having the longitudinal axis O1 as its center axis. An accommodation space 27 penetrating the main body portion 21 in the direction Y1 is formed between the distal end portion 21A and a proximal end portion 21B of the main body portion 21. The connection member 15A of the first gripping piece 11A is fixed to a part 21Aa of the distal end portion 21A on the direction Y1 side. A cut-out portion 21C is formed at a part 21Ab of the distal end portion 21A on the direction Y2 side. The cut-out portion 21C penetrates the distal end portion 21A in a direction along the longitudinal axis O1 and communicates with the accommodation space 27. The cut-out portion 21C is open in the direction Y2 and is formed to extend in the direction Y1 beyond the axis O20A. An end surface 21Ca of the cut-out portion 21C on the direction Y1 side is formed to be directed in the direction Y1 from the distal end side toward the proximal end side. The end surface 21Ca regulates a pivot range of the second gripping piece 11B when the end surface 21Ca comes into contact with a side surface 16B of the gear portion 16 on the direction Y1 side. The dimensions of the cut-out portion 21C in the direction X1 are set to be greater than the dimensions of the connection member 15B and the gear portion 16 of the second gripping piece 11B in the direction X1. In addition, penetration holes having the axis O20A as their center axis are formed in the distal end portion 21A on both sides in the direction X1 with the cut-out portion 21C interposed therebetween. Both ends of the pivot axis 22 are pivotably supported by the penetration holes.

The accommodation space 27 is formed into a rectangular shape when seen in the direction Y1. The gear portion 24, the pulley 25, the pulley 26, and the gear portion 16 of the second gripping piece 11B are arranged in the accommodation space 27. A penetration hole having an axis O20B as its center axis is formed in a side portion 21E of the main body portion 21 (a side wall portion of the accommodation space 27 on the direction X1 side) orthogonal to the longitudinal axis O1 and parallel to the direction X1. In addition, a penetration hole having the axis O20B as its center axis is formed in a side portion 21F of the main body portion 21 (a side wall portion of the accommodation space 27 on the direction X2 side). Both ends of a pivot axis 23 formed into a columnar shape extending in the direction X1 and having the axis O20B as its center axis are respectively inserted through the penetration hole of the side portion 21E and the penetration hole of the side portion 21F and are pivotably supported therein.

A circular hole 24A penetrating the gear portion 24 in the direction X1 about the axis O20B is formed in the gear portion 24. The pivot axis 23 is relatively and pivotably inserted through the circular hole 24A around the axis O20B. The gear portion 24 is formed into a columnar shape about the axis O20B. Teeth are formed on an outer circumferential surface around the axis O20B of the gear portion 24. Since the gear portion 24 engages with the gear portion 16 of the second gripping piece 11B, the second gripping piece 11B can pivot around the axis O20A by causing the gear portion 24 to pivot around the axis O20B.

The pulley 25 is formed into a disk shape having the axis O20B as its center axis and has a circular hole (not shown) penetrating the pulley 25 in the direction X1 about the axis O20B. In the pulley 25, a pulley groove 25A is formed on the outer circumferential surface around the axis O20B. In addition, the pulley 26 has a similar configuration to that of the pulley 25. The pivot axis 23 is relatively and pivotably inserted through the circular hole of the pulley 25 and the circular hole of the pulley 26 around the axis O20B. The pulley 25 is provided to be adjacent to the gear portion 24 in the direction X1 and is fixed to the gear portion 24 to pivot together with the gear portion 24. The pulley 26 is provided to be adjacent to the gear portion 24 in the direction X2 and is fixed to the gear portion 24 to pivot together with the gear portion 24.

An operation wire 25W is wound around the pulley groove 25A of the pulley 25. One end of the operation wire 25W is fixed to the pulley groove 25A by a fixing portion (not shown). The other end of the operation wire 25W is connected to a power-transmitting portion (not shown) provided on the proximal end side of the medical instrument 1. The operation wire 25W is wound counterclockwise around the pulley groove 25A when seen in the direction X2 from the other end toward one end of the operation wire 25W. In addition, an operation wire 26W is wound around a pulley groove 26A of the pulley 26. One end of the operation wire 26W is fixed to the pulley groove 26A by a fixing portion 26B. The other end of the operation wire 26W is connected to a power-transmitting portion. The operation wire 26W is wound clockwise around the pulley groove 26A when seen in the direction X2 from the other end toward one end of the operation wire 26W.

With the configuration described above, when the operation wire 25W is pulled to the proximal end side, the gear portion 24 pivots clockwise together with the pulley 25 when seen in the direction X2. Incidental to this pivoting of the gear portion 24, the gear portion 16 which has engaged with the gear portion 24 pivots counterclockwise together with the second gripping piece 11B when seen in the direction X2. Accordingly, the pair of gripping pieces 11 is open. In addition, when the operation wire 26W is pulled to the proximal end side, the gear portion 24 pivots counterclockwise together with the pulley 26 when seen in the direction X2. Incidental to this pivoting of the gear portion 24, the gear portion 16 pivots clockwise together with the second gripping piece 11B when seen in the direction X2. Accordingly, the pair of gripping pieces 11 is closed. In this manner, the pair of gripping pieces 11 makes opening and closing movements in accordance with operations of the operation wire 25W and the operation wire 26W.

A cut-out portion 21Ha and a cut-out portion 21Hb through which the operation wire 25W and the operation wire 26W respectively pass are formed in the proximal end portion 21B of the main body portion 21. The cut-out portion 21Ha is formed at a part on the direction X1 side in the end portion of the proximal end portion 21B on the direction Y1 side. The operation wire 25W passes through the cut-out portion 21Ha. In addition, the cut-out portion 21Hb is formed at a part on the direction X2 side in the end portion of the proximal end portion 21B on the direction Y2 side. The operation wire 26W passes through the cut-out portion 21Hb.

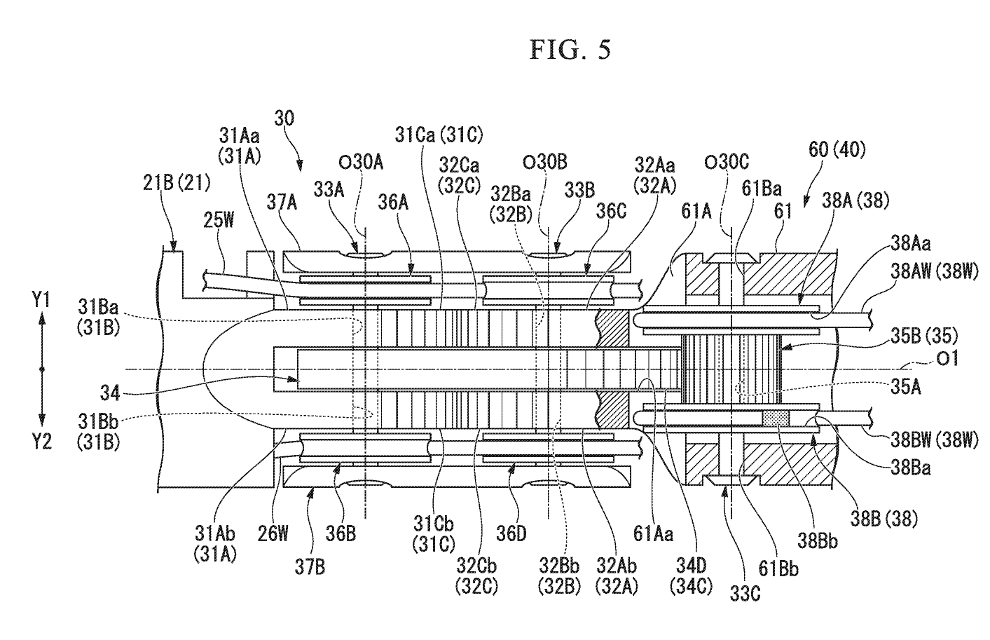

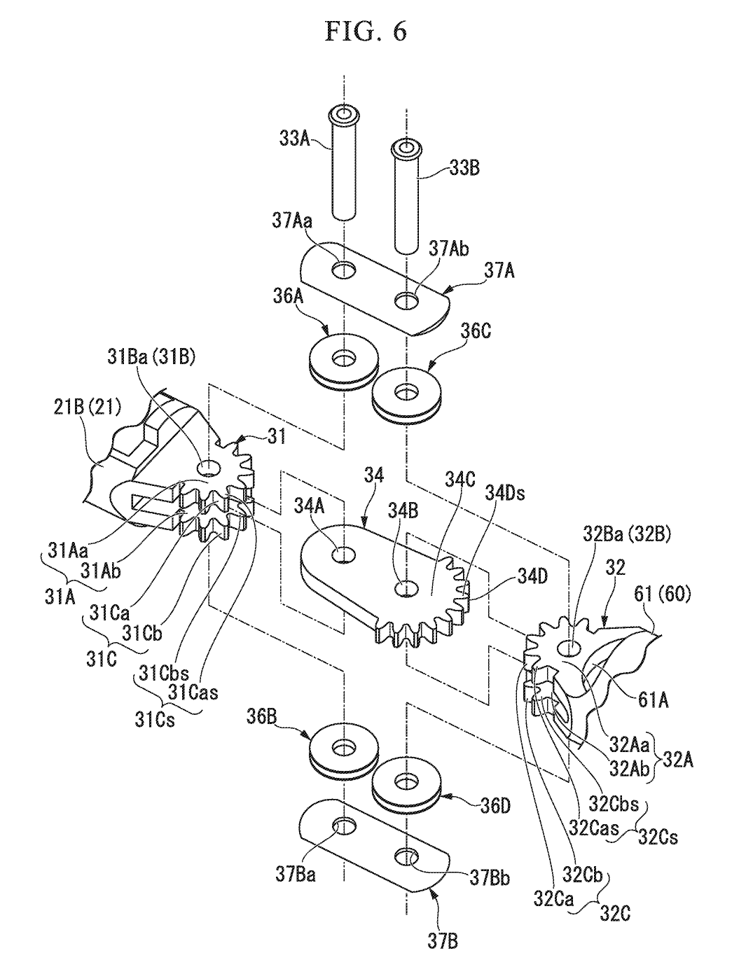

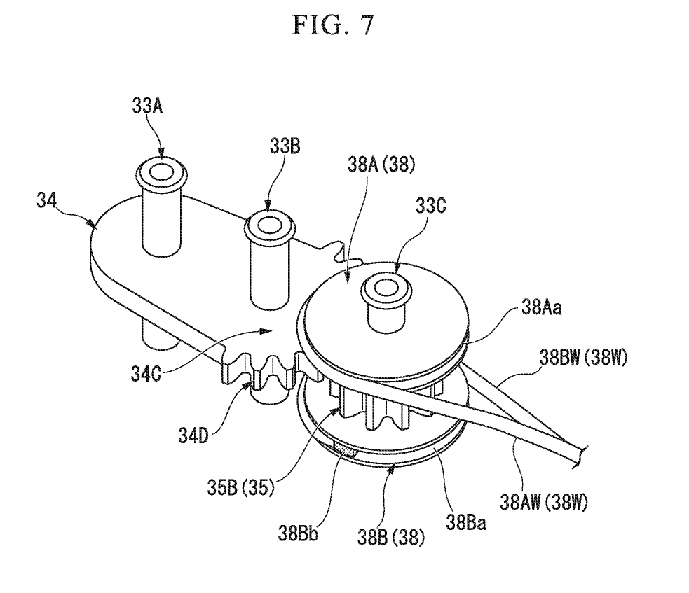

FIG. 5 is a part of a cross-sectional view illustrating a configuration of the distal end joint 30 of the medical instrument 1. FIG. 6 is an exploded perspective view of the distal end joint 30. FIG. 7 is a perspective view illustrating a configuration of an intermediate connection member 34 and a rotation-transmitting portion 35 of the distal end joint 30. The distal end joint 30 is a double joint articulation which is bent around the direction Y1. The distal end joint 30 has an axis (first axis) O30A, an axis (second axis) O30B, and an axis (third axis) O30C. The axis O30B is provided to be separated from the axis O30A and is arranged to be parallel to the axis O30A. In addition, the axis O30B is arranged on the proximal end side of the axis O30A. The axis O30C is provided to be separated from the axis O30A and the axis O30B and is arranged to be parallel to the axis O30A and the axis O30B. In addition, the axis O30C is arranged on the proximal end side of the axis O30B and its position is fixed with respect to the axis O30B. In the present embodiment, the axis O30A, the axis O30B, and the axis O30C are orthogonal to the longitudinal axis O1 and are parallel to the direction Y1.

In addition, the distal end joint 30 has a distal end connection member (first connection member) 31, a proximal end connection member (second connection member) 32, an intermediate connection member (third connection member) 34, and a rotation-transmitting portion (third rotation-transmitting portion) 35.

The distal end connection member 31 is fixed to the proximal end side of the proximal end portion 21B of the main body portion 21 (refer to FIG. 4). The distal end connection member 31 has a rotation-transmitting portion (first rotation-transmitting portion) 31A provided on the proximal end side. The rotation-transmitting portion 31A is configured to be pivotable about the axis O30A. A circular hole 31B penetrating the rotation-transmitting portion 31A in the direction Y1 about the axis O30A is formed in the rotation-transmitting portion 31A. A pivot axis 33A formed into a columnar shape extending in the direction Y1 and having the axis O30A as its center axis is inserted through the circular hole 31B. The pivot axis 33A is relatively and pivotably supported by the rotation-transmitting portion 31A via the circular hole 31B about the axis O30A. That is, the rotation-transmitting portion 31A is pivotable about the axis O30A with respect to the pivot axis 33A.

The rotation-transmitting portion 31A is divided into an upper rotation-transmitting portion (first part) 31Aa and a lower rotation-transmitting portion (second part) 31Ab in a direction along the axis O30A, that is, in the direction Y1 (or the direction Y2). The upper rotation-transmitting portion 31Aa and the lower rotation-transmitting portion 31Ab are separated from each other in the direction Y1 and are arranged to be parallel to each other with the longitudinal axis O1 interposed therebetween. The upper rotation-transmitting portion 31Aa is arranged on the direction Y1 side of the longitudinal axis O1, and the lower rotation-transmitting portion 31Ab is arranged on the direction Y2 side of the longitudinal axis O1. In addition, as the circular hole 31B, a circular hole 31Ba penetrating the upper rotation-transmitting portion 31Aa in the direction Y1 about the axis O30A is formed in the upper rotation-transmitting portion 31Aa, and a circular hole 31Bb penetrating the lower rotation-transmitting portion 31Ab in the direction Y1 about the axis O30A is formed in the lower rotation-transmitting portion 31Ab. The pivot axis 33A is inserted through the circular hole 31Ba and the circular hole 31Bb.

The rotation-transmitting portion 31A includes a gear portion (first gear portion) 31C which has a sector-shaped site (first site) 31Cs about the axis O30A and in which teeth are formed on an arc of the sector-shaped site 31Cs. As the gear portion 31C, the upper rotation-transmitting portion 31Aa includes an upper gear portion (first gear portion) 31Ca which has a sector-shaped site (first site) 31Cas about the axis O30A and in which teeth are formed on an arc of the sector-shaped site 31Cas. In addition, the lower rotation-transmitting portion 31Ab includes a lower gear portion (first gear portion) 31Cb which has a sector-shaped site (first site) 31Cbs about the axis O30A and in which teeth are formed on an arc of the sector-shaped site 31Cbs. The gear portion 31C is configured to engage with a gear portion (second gear portion) 32C of a rotation-transmitting portion 32A (which will be described below). Specifically, the upper gear portion 31Ca engages with an upper gear portion 32Ca of the gear portion 32C, and the lower gear portion 31Cb engages with a lower gear portion 32Cb of the gear portion 32C. Consequently, the rotation-transmitting portion 31A and the rotation-transmitting portion 32A are connected to each other to pivot by interlocking with each other.

The proximal end connection member 32 is provided on the distal end side of the intermediate arm 40 and is fixed to the intermediate arm 40. In the present embodiment, the proximal end connection member 32 is fixed to a distal end portion 61 of the arm 60 in the intermediate arm 40. The proximal end connection member 32 has a rotation-transmitting portion (second rotation-transmitting portion) 32A provided on the distal end side. The rotation-transmitting portion 32A is configured to be pivotable about the axis O30B. Specifically, similar to the rotation-transmitting portion 31A, a pivot axis 33B formed into a columnar shape extending in the direction Y1 and having the axis O30B as its center axis is inserted through a circular hole 32B penetrating the rotation-transmitting portion 32A in the direction Y1 about the axis O30B formed in the rotation-transmitting portion 32A and is pivotably supported with respect to the rotation-transmitting portion 32A.

The rotation-transmitting portion 32A is divided into an upper rotation-transmitting portion (third part) 32Aa and a lower rotation-transmitting portion (fourth part) 32Ab in a direction along the axis O30B, that is, in the direction Y1 (or the direction Y2). The upper rotation-transmitting portion 32Aa and the lower rotation-transmitting portion 32Ab are arranged in a form similar to that of the upper rotation-transmitting portion 31Aa and the lower rotation-transmitting portion 31Ab of the rotation-transmitting portion 31A. In addition, as the circular hole 32B, a circular hole 32Ba is formed in the upper rotation-transmitting portion 32Aa, and a circular hole 32Bb is formed in the lower rotation-transmitting portion 32Ab.

The rotation-transmitting portion 32A includes the gear portion (second gear portion) 32C which has a sector-shaped site (second site) 32Cs about the axis O30B and in which teeth are formed on an arc of the sector-shaped site 32Cs. As the gear portion 32C, the upper rotation-transmitting portion 32Aa has an upper gear portion (second gear portion) 32Ca, and the lower rotation-transmitting portion 32Ab has a lower gear portion (second gear portion) 32Cb. As the sector-shaped site 32Cs, the upper gear portion 32Ca has a sector-shaped site (second site) 32Cas, and the lower gear portion 32Cb has a sector-shaped site (second site) 32Cbs.

In the present embodiment, the pitch circle diameter of the gear portion 31C (upper gear portion 31Ca and lower gear portion 31Cb) is the same as the pitch circle diameter of the gear portion 32C (upper gear portion 32Ca and lower gear portion 32Cb). In addition, the dimensions of the gear portion 31C in the direction Y1 are the same as the dimensions of the gear portion 32C in the direction Y1. That is, the dimensions of the upper gear portion 31Ca in the direction Y1 and the dimensions of the upper gear portion 32Ca in the direction Y1 are equal to each other, and the dimensions of the lower gear portion 31Cb in the direction Y1 and the dimensions of the lower gear portion 32Cb in the direction Y1 are equal to each other.

The intermediate connection member 34 is configured to be pivotable about the axis O30A and to be pivotable about the axis O30B. In the present embodiment, the intermediate connection member 34 is a plate-shaped member having a substantially elliptical shape when seen in the direction Y1. A circular hole 34A penetrating the intermediate connection member 34 in the direction Y1 about the axis O30A is formed on the distal end side of the intermediate connection member 34. The pivot axis 33A is inserted through the circular hole 34A. The pivot axis 33A is relatively and pivotably supported by the intermediate connection member 34 via the circular hole 34A about the axis O30A. That is, the intermediate connection member 34 is pivotable about the axis O30A with respect to the pivot axis 33A. In addition, a circular hole 34B penetrating the intermediate connection member 34 in the direction Y1 about the axis O30B is formed on the proximal end side of the intermediate connection member 34. The pivot axis 33B is inserted through the circular hole 34B. The pivot axis 33B is relatively and pivotably supported by the intermediate connection member 34 via the circular hole 34B about the axis O30B. That is, the intermediate connection member 34 is pivotable about the axis O30B relative to the pivot axis 33B.

In the present embodiment, the distal end side of the intermediate connection member 34 is arranged between the upper rotation-transmitting portion 31Aa and the lower rotation-transmitting portion 31Ab of the rotation-transmitting portion 31A. The proximal end side of the intermediate connection member 34 is arranged between the upper rotation-transmitting portion 32Aa and the lower rotation-transmitting portion 32Ab of the rotation-transmitting portion 32A. Thus, the intermediate connection member 34 is arranged so as to include the longitudinal axis O1. In addition, the dimensions of the intermediate connection member 34 on the direction Y1 are smaller than the dimensions between the upper rotation-transmitting portion 31Aa and the lower rotation-transmitting portion 31Ab and the dimensions between the upper rotation-transmitting portion 32Aa and the lower rotation-transmitting portion 32Ab.

The intermediate connection member 34 has a rotation-transmitting portion (fourth rotation-transmitting portion) 34C provided in the proximal end portion (end portion) of the intermediate connection member 34. The rotation-transmitting portion 34C is connected to the rotation-transmitting portion 35 to pivot about the axis O30B by interlocking with the rotation-transmitting portion 35. In the present embodiment, the rotation-transmitting portion 34C includes a gear portion (third gear portion) 34D which has a sector-shaped site (third site) 34Ds about the axis O30B and in which teeth are formed on an arc of the sector-shaped site 34Ds. The gear portion 34D is configured to engage with a gear portion (fourth gear portion) 35B of the rotation-transmitting portion 35 (which will be described below). Therefore, rotational motion of the rotation-transmitting portion 35 is transmitted to the rotation-transmitting portion 34C via the gear portion 35B and the gear portion 34D. Consequently, the rotation-transmitting portion 34C can pivot about the axis O30B by interlocking with the rotation-transmitting portion 35.

A pulley 36A and a pulley 36B are provided on both sides of the rotation-transmitting portion 31A in the direction Y1. The pulley 36A is formed into a disk shape having the axis O30A as its center axis and has a circular hole penetrating the pulley 36A in the direction Y1 about the axis O30A. In the pulley 36A, a pulley groove is formed on the outer circumferential surface around the axis O30A. In addition, the pulley 36B has a similar configuration to that of the pulley 36A.

The pivot axis 33A is relatively and pivotably inserted through the circular hole of the pulley 36A and the circular hole of the pulley 36B around the axis O30A. The pulley 36A is arranged on the direction Y1 side of the upper rotation-transmitting portion 31Aa, and the pulley 36B is arranged on the direction Y2 side of the lower rotation-transmitting portion 31Ab. Thus, the rotation-transmitting portion 31A is arranged between the pulley 36A and the pulley 36B in the direction Y1. The operation wire 25W extending from the pulley 25 is wound around the pulley groove of the pulley 36A. The operation wire 26W extending from the pulley 26 is wound around the pulley groove of the pulley 36B.

A pulley 36C and a pulley 36D are provided on both sides of the rotation-transmitting portion 32A in the direction Y1. The pulley 36C and the pulley 36D have a similar configuration to that of the pulley 36A, excluding that the axis O30B serves as the center axis instead of the axis O30A.

The pivot axis 33B is relatively and pivotably inserted through the circular hole of the pulley 36C and the circular hole of the pulley 36D around the axis O30B. The pulley 36C is arranged on the direction Y1 side of the upper rotation-transmitting portion 32Aa, and the pulley 36D is arranged on the direction Y2 side of the lower rotation-transmitting portion 32Ab. Thus, the rotation-transmitting portion 32A is arranged between the pulley 36C and the pulley 36D in the direction Y1. The operation wire 25W extending from the pulley 36A is wound around the pulley groove of the pulley 36C. The operation wire 26W extending from the pulley 36B is wound around the pulley groove of the pulley 36D.

The pulley 36A and the pulley 36C are arranged at the same positions in the direction Y1. Similarly, the pulley 36B and the pulley 36D are arranged at the same positions in the direction Y1. The operation wire 25W is wound around from the direction X1 side of the pulley 36A to the direction X2 side of the pulley 36C in a manner intersecting the longitudinal axis O1 between the pulley 36A and the pulley 36C when seen in the direction Y2. The operation wire 26W is wound around from the direction X2 side of the pulley 36B to the direction X1 side of the pulley 36D in a manner intersecting the longitudinal axis O1 between the pulley 36B and the pulley 36D when seen in the direction Y2.

A support member 37A is provided on the direction Y1 side of the pulley 36A and the pulley 36C. The support member 37A is a plate-shaped member having a substantially elliptical shape when seen in the direction Y1. A circular hole 37Aa penetrating the support member 37A in the direction Y1 about the axis O30A and a circular hole 37Ab penetrating the support member 37A in the direction Y1 about the axis O30B are formed in the support member 37A. The pivot axis 33A is relatively and pivotably inserted through the circular hole 37Aa about the axis O30A. The pivot axis 33B is relatively and pivotably inserted through the circular hole 37Ab about the axis O30B.

A support member 37B is provided on the direction Y2 side of the pulley 36B and the pulley 36D. The support member 37B has the same configuration as that of the support member 37A. The pivot axis 33A is relatively and pivotably inserted through a circular hole 37Ba of the support member 37B about the axis O30A. The pivot axis 33B is relatively and pivotably inserted through a circular hole 37Bb of the support member 37B about the axis O30B.

Both ends of the pivot axis 33A are respectively supported by the support member 37A and the support member 37B via the circular hole 37Aa and the circular hole 37Ba. Similarly, both ends of the pivot axis 33B are respectively supported by the support member 37A and the support member 37B via the circular hole 37Ab and the circular hole 37Bb.

With the configuration described above, the pivot axis 33A relatively and pivotably connects the support member 37A, the pulley 36A, the rotation-transmitting portion 31A (upper rotation-transmitting portion 31Aa and lower rotation-transmitting portion 31Ab), the intermediate connection member 34, the pulley 36B, and the support member 37B to each other about the axis O30A. Similarly, the pivot axis 33B relatively and pivotably connects the support member 37B, the pulley 36C, the rotation-transmitting portion 32A (upper rotation-transmitting portion 32Aa and lower rotation-transmitting portion 32Ab), the intermediate connection member 34, the pulley 36D, and the support member 37B to each other about the axis O30B. In addition, the distance between the axis O30A and the axis O30B is uniformly retained by the support member 37A, the intermediate connection member 34, and the support member 37B.

The rotation-transmitting portion 35 is configured to be pivotable about the axis O30C. Specifically, a circular hole 35A penetrating the rotation-transmitting portion 35 in the direction Y1 about the axis O30C is formed. A pivot axis 33C formed into a columnar shape extending in the direction Y1 and having the axis O30C as its center axis is inserted through the circular hole 35A. The pivot axis 33C is relatively and pivotably supported by the rotation-transmitting portion 35 via the circular hole 35A about the axis O30C. That is, the rotation-transmitting portion 35 is pivotable about the axis O30C with respect to the pivot axis 33C.

The rotation-transmitting portion 35 has the gear portion (fourth gear portion) 35B which is formed into a columnar shape about the axis O30C and in which teeth are formed on the outer circumferential surface around the axis O30C. As described above, the gear portion 35B engages with the gear portion 34D. In addition, the pitch circle diameter of the gear portion 35B is smaller than the pitch circle diameter of the gear portion 34D. Therefore, the rotational frequency of the gear portion 34D can be lower than the rotational frequency of the gear portion 35B, and the torque of the gear portion 34D can be higher than the torque of the gear portion 35B.

In addition, the distal end joint 30 has a pulley (rotor) 38 which is pivotable about the axis O30C and an operation wire (operation member) 38W which is wound around the pulley 38. The pulley 38 is connected to the gear portion 35B to integrally pivot with the gear portion 35B. In the present embodiment, as the pulley 38, a pulley (rotor) 38A and a pulley (rotor) 38B are provided. As the operation wire 38W, an operation wire (operation member) 38AW and an operation wire (operation member) 38BW are provided.

The pulley 38A is formed into a disk shape having the axis O30C as its center axis and has a circular hole (not shown) penetrating the pulley 38A in the direction Y1 about the axis O30C. In the pulley 38A, a pulley groove 38Aa is formed on the outer circumferential surface around the axis O30C. The pulley 38B has a similar configuration to that of the pulley 38A. The pivot axis 33C is relatively and pivotably inserted through the circular hole of the pulley 38A and the circular hole of the pulley 38B around the axis O30C. The pulley 38A is provided to be adjacent to the gear portion 35B in the direction Y1 and is fixed to the gear portion 35B to pivot together with the gear portion 35B. The pulley 38B is provided to be adjacent to the gear portion 35B in the direction Y2 and is fixed to the gear portion 35B to pivot together with the gear portion 35B.

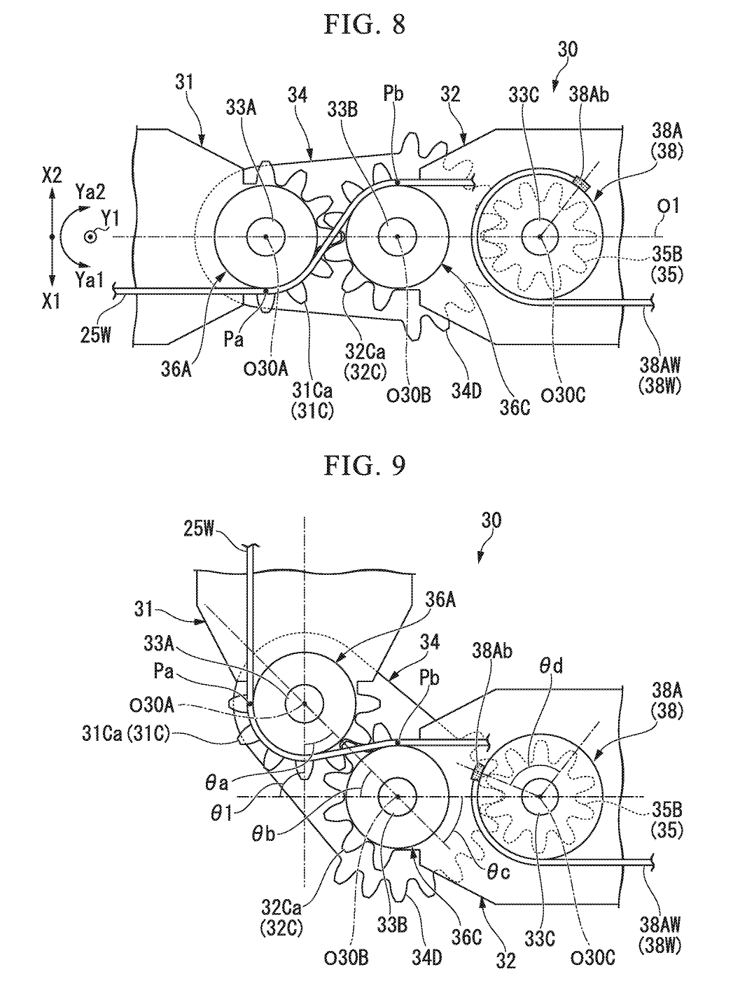

The operation wire 38AW is wound around the pulley groove 38Aa of the pulley 38A. One end of the operation wire 38AW is fixed to the pulley groove 38Aa by a fixing portion 38Ab (refer to FIG. 8). The other end of the operation wire 38AW is connected to a power-transmitting portion (not shown) provided on the proximal end side of the medical instrument 1. The operation wire 38AW is wound counterclockwise around the pulley groove 38Aa when seen in the direction Y2 from the other end toward one end of the operation wire 38AW. In addition, the operation wire 38BW is wound around a pulley groove 38Ba of the pulley 38B. One end of the operation wire 38BW is fixed to the pulley groove 38Ba by a fixing portion 38Bb. The other end of the operation wire 38BW is connected to a power-transmitting portion (not shown) provided on the proximal end side of the medical instrument 1. The operation wire 38BW is wound clockwise around the pulley groove 38Ba when seen in the direction Y2 from the other end toward one end of the operation wire 38BW.

With the configuration described above, when the operation wire 38AW is pulled to the proximal end side, the gear portion 35B pivots counterclockwise together with the pulley 38A when seen in the direction Y2. In addition, when the operation wire 38BW is pulled to the proximal end side, the pulley 38B pivots clockwise together with the gear portion 35B when seen in the direction Y2.

In the present embodiment, the outer diameter of the pulley 38A and the pulley 38B is larger than the pitch circle diameter of the gear portion 35B. Accordingly, it is possible to reduce a tensile force of the operation wire 38AW and the operation wire 38BW required for the gear portion 35B to pivot.

The arm 60 is formed into a cylindrical shape having the longitudinal axis O1 as its center axis. An accommodation space 61A extending along the longitudinal axis O1 is formed inside the distal end portion 61 of the arm 60. The accommodation space 61A is arranged on the proximal end side of the proximal end connection member 32 fixed to the distal end portion 61. The accommodation space 61A communicates with a space formed between the upper rotation-transmitting portion 32Aa and the lower rotation-transmitting portion 32Ab of the rotation-transmitting portion 32A via an opening 61Aa in a direction along the longitudinal axis O1. In addition, a circular hole 61Ba and a circular hole 61Bb penetrating the distal end portion 61 about the axis O30C are formed in the distal end portion 61. The circular hole 61Ba and the circular hole 61Bb individually communicate with the accommodation space 61A and are arranged with the accommodation space 61A interposed therebetween.

The rotation-transmitting portion 35, the pulley 38A, and the pulley 38B are arranged on the accommodation space 61A. The gear portion 34D of the rotation-transmitting portion 34C passes through the opening 61Aa, is arranged inside the accommodation space 61A, and engages with the gear portion 35B of the rotation-transmitting portion 35. Both ends of the pivot axis 33C are relatively and pivotably supported by the circular hole 61Ba and the circular hole 61Bb about the axis O30C. The pivot axis 33C is supported by the distal end portion 61 through the circular hole 61Ba and the circular hole 61Bb, and the pivot axis 33B is supported by the rotation-transmitting portion 32A of the proximal end connection member 32 fixed to the distal end portion 61. Therefore, the position of the axis O30C is fixed with respect to the axis O30B.

Movements of the distal end joint 30 having the configuration described above will be described with reference to FIG. 8 and FIG. 9. FIG. 8 is a diagram illustrating a state where the distal end joint 30 is straight. FIG. 9 is a diagram illustrating a state where the distal end joint 30 is bent. FIG. 8 and FIG. 9 are diagrams of the distal end joint 30 seen in the direction Y2. In FIG. 8 and FIG. 9, the configuration of the distal end joint 30 is shown in a simplified manner. In addition, as shown in FIG. 8 and FIG. 9, as rotation directions around the direction Y1, a direction Ya1 and a direction Ya2 opposite to the direction Ya1 are set.

As shown in FIG. 8, when the operation wire 38AW is pulled to the proximal end side in a state where the distal end joint 30 is straight, the gear portion 35B fixed to the pulley 38A pivots counterclockwise, that is, about the axis O30C in the direction Ya1 when seen in the direction Y2. Since the gear portion 35B engages with the gear portion 34D of the intermediate connection member 34, the gear portion 34D pivots about the axis O30B in the direction Ya2 incidental to pivoting of the gear portion 35B. That is, the intermediate connection member 34 pivots about the axis O30B in the direction Ya2 with respect to the proximal end connection member 32. In this case, the pivot axis 33A, that is, the axis O30A also rotatively moves about the axis O30B in the direction Ya2 together with the intermediate connection member 34. In addition, since the gear portion 31C of the distal end connection member 31 engages with the gear portion 32C of the proximal end connection member 32, the gear portion 31C pivots about the axis O30A in the direction Ya2 incidental to pivoting of the intermediate connection member 34. That is, the distal end connection member 31 pivots about the axis O30A in the direction Ya2 with respect to the intermediate connection member 34. Due to the movement described above, as shown in FIG. 9, the distal end joint 30 is in a state of being bent in the direction Ya2. In a case where the distal end joint 30 is to be in a straight state from this state, or in a case where the distal end joint 30 is to be bent in the direction Ya1 from a straight state, the operation wire 38BW is pulled to the proximal end side. Accordingly, the gear portion 35B fixed to the pulley 38B pivots about the axis O30C in the direction Ya2 opposite to the movement described above. Then, each of the members makes a movement in a direction opposite to that of the movement described above, so that the distal end joint 30 is bent in the direction Ya1.

Bending movement of the above-described distal end joint 30 will be described in more detail. Here, the radius of a pitch circle of the gear portion 31C is Ra, the radius of a pitch circle of the gear portion 32C is Rb, the radius of a pitch circle of the gear portion 34D is Rc, and the radius of a pitch circle of the gear portion 35B is Rd. When the gear portion 35B pivots about the axis O30C in the direction Ya1 by an angle θd, in a case where the gear portion 34D pivots about the axis O30B in the direction Ya2 with respect to the proximal end connection member 32 by an angle θc, the angle θc is expressed by the following expression.

θc=θd×Rd/Rc (1)

In this case, when an angle at which the gear portion 31C pivots about the axis O30A in the direction Ya2 with respect to the intermediate connection member 34 is Oa and an angle at which the gear portion 32C pivots about the axis O30B in the direction Ya1 with respect to the intermediate connection member 34 is θb, the angle θa is expressed by the following expression.

θa=θb×Rb/Ra (2)

In the present embodiment, the pitch circle diameter of the gear portion 31C and the pitch circle diameter of the gear portion 32C are equal to each other. In addition, the size of the angle θb and the size of the angle θc are the same as each other. Thus, Expression (2) is established as follows.

θa=θb=θc (3)

Therefore, an angle θ1 at which the distal end connection member 31 pivots with respect to the proximal end connection member 32 is expressed by the following expression.

θ1=θa+θc=2θc (4)

That is, the angle θ1 at which the distal end connection member 31 pivots with respect to the proximal end connection member 32 becomes twice the angle θc at which the intermediate connection member 34 pivots with respect to the proximal end connection member 32.

In addition, when the torque while the gear portion 34D pivots is Tc and the torque while the gear portion 35B pivots is Td, the torque Tc is expressed by the following expression.

Tc=Td×Rc/Rd (5)

In the present embodiment, since the pitch circle diameter of the gear portion 35B is smaller than the pitch circle diameter of the gear portion 34D, the relationship of Rc/Rd (reduction ratio)>1 is established. The torque Td of the gear portion 35B can be reduced with respect to the torque Tc of the gear portion 34D by increasing the reduction ratio in this manner. Thus, it is possible to reduce a tensile force applied to the operation wire 38AW and the operation wire 38BW when the gear portion 35B pivots.

In the double joint articulation, bending of the articulation does not affect a path length of the operation wire. For example, in the operation wire 25W wound around the pulley 36A and the pulley 36C, the path length between a point Pa and a point Pb shown in FIG. 8 is equal to the path length between the point Pa and the point Pb in a state where the distal end joint 30 is bent as shown in FIG. 9. Therefore, the distal end joint 30 can be bent in a manner independent of an operation of the pair of gripping pieces 11.

In the configuration described above, the distal end joint 30 is resistant to an external disturbance in a case where an external force of tilting the axis of the articulation acts. For example, in a case where a force of the pair of gripping pieces 11 gripping a target is significant, a tensile force applied to the operation wire 26W also becomes significant. Due to this tensile force, a force of tilting the pivot axis 33A and the pivot axis 33B to widen the gap between the pivot axis 33A and the pivot axis 33B acts on the pivot axis 33A and the pivot axis 33B via the pulley 36B and the pulley 36D around which the operation wire 26W is wound. In a case where the pivot axis 33A and the pivot axis 33B are tilted and are arranged in a positional relationship of non-equilibrium, since the gear portion 31C and the gear portion 32C engage with each other in a tilted state, a frictional force between the gear portions increases. As a result, controllability or operability of the end effector 10 deteriorates.

In the double joint articulation in the related art, mainly, a gear portion of the distal end connection member through which one pivot axis is inserted and a gear portion of the proximal end connection member through which the other pivot axis is inserted engage with each other and are supported. Accordingly, the orientation of the pivot axes is maintained against a force acting on the pivot axes as described above and a moment caused by this force. Moreover, following the points described above, in the distal end joint 30 according to the present embodiment, the rotation-transmitting portion 31A of the distal end connection member 31 is divided into the upper rotation-transmitting portion 31Aa having the upper gear portion 31Ca and the lower rotation-transmitting portion 31Ab having the lower gear portion 31Cb. The rotation-transmitting portion 32A of the proximal end connection member 32 is divided into the upper rotation-transmitting portion 32Aa having the upper gear portion 32Ca and the lower rotation-transmitting portion 32Ab having the lower gear portion 32Cb. The intermediate connection member 34 is arranged between the upper rotation-transmitting portion 31Aa and the lower rotation-transmitting portion 31Ab and is arranged between the upper rotation-transmitting portion 32Aa and the lower rotation-transmitting portion 32Ab. Accordingly, the dimensions of the gear portion 31C of the rotation-transmitting portion 31A in the direction Y1 and the dimensions of the gear portion 32C of the rotation-transmitting portion 32A in the direction Y1 can be practically increased. That is, the upper gear portion 31Ca and the upper gear portion 32Ca move the direction Y1 side and are arranged thereon in accordance with the dimensions of a space in the direction Y1, in which the intermediate connection member 34 is arranged. The lower gear portion 31Cb and the lower gear portion 32Cb move to the direction Y2 side and are arranged thereon in accordance with the dimensions of the space. As a result, the dimensions of the gear portion 31C in its entirety in the direction Y1 and the dimensions of the gear portion 32C in its entirety in the direction Y1 have increased. In addition, the upper gear portion 31Ca and the upper gear portion 32Ca engage with each other at a position closer to the end portion of the pivot axis 33A and the pivot axis 33B in the direction Y1. The lower gear portion 31Cb and the lower gear portion 32Cb engage with each other at a position closer to the end portion of the pivot axis 33A and the pivot axis 33B in the direction Y2. Thus, due to the gear portion 31C and the gear portion 32C, the orientation of the pivot axis 33A and the pivot axis 33B can be more reliably maintained against the moment acting on the pivot axis 33A and the pivot axis 33B by the above-described force. In this manner, the distal end joint 30 has a configuration resistant to an external disturbance.

In addition, in the present embodiment, the intermediate connection member 34 is arranged between the upper rotation-transmitting portion 31Aa and the lower rotation-transmitting portion 31Ab and is arranged between the upper rotation-transmitting portion 32Aa and the lower rotation-transmitting portion 32Ab. At the same time, the intermediate connection member 34 supports a middle part of the pivot axis 33A and the pivot axis 33B in the direction Y1. Therefore, it is possible to reduce the moment acting on the pivot axis 33A to be tilted with respect to the pivot axis 33B by a force which the intermediate connection member 34 applies to the pivot axis 33A to move the pivot axis 33A, when the intermediate connection member 34 pivots about the axis O30B due to the gear portion 35B.

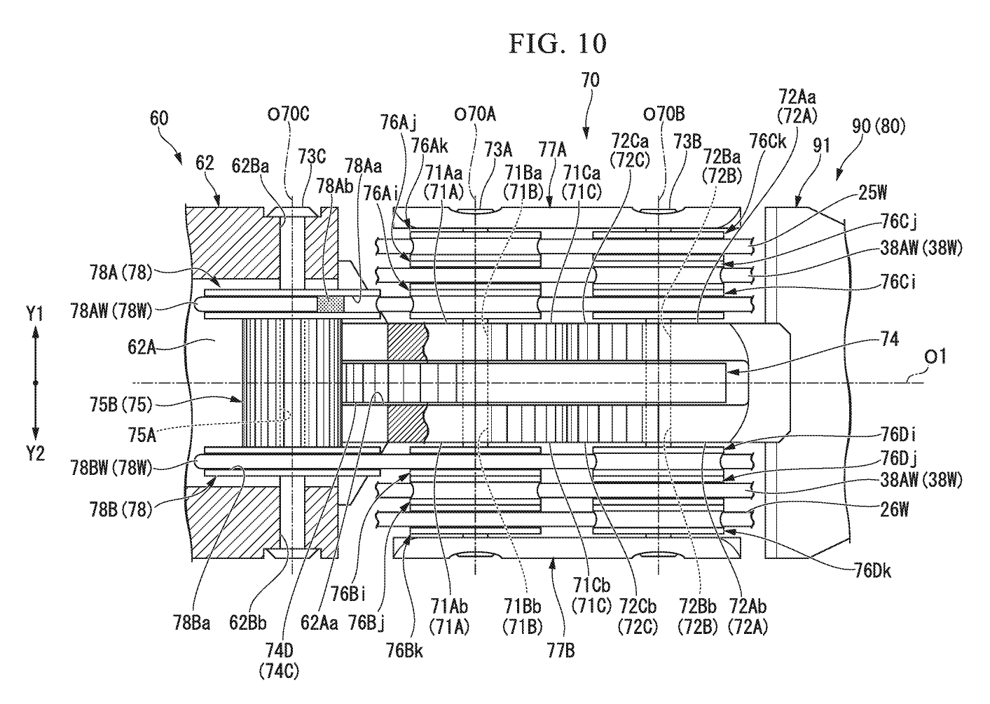

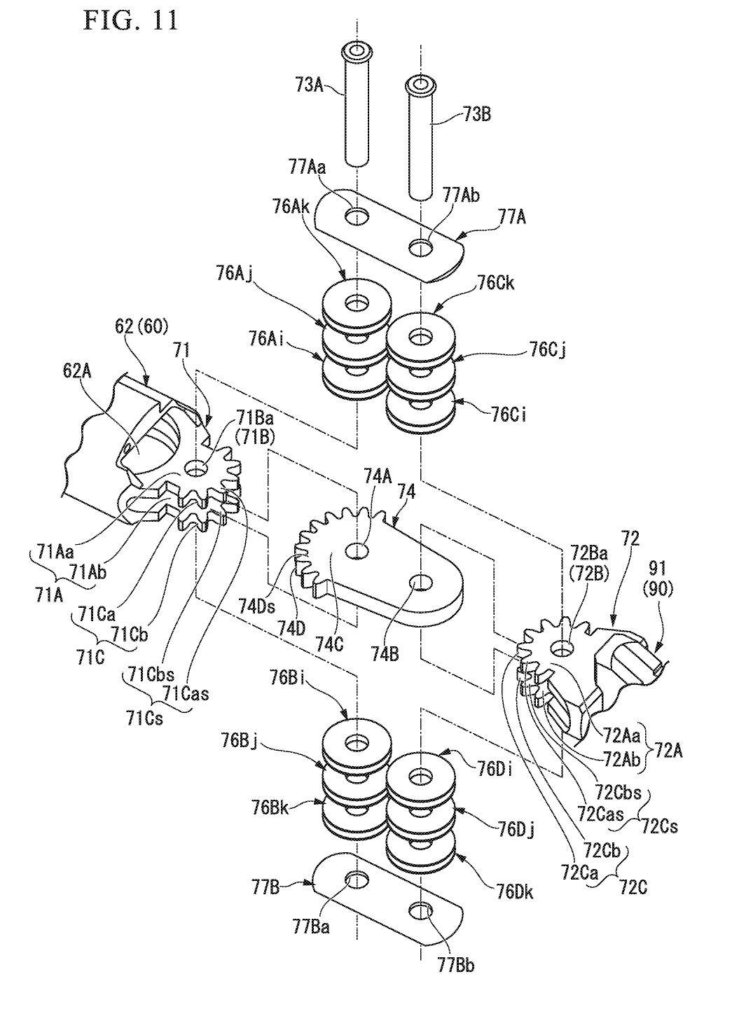

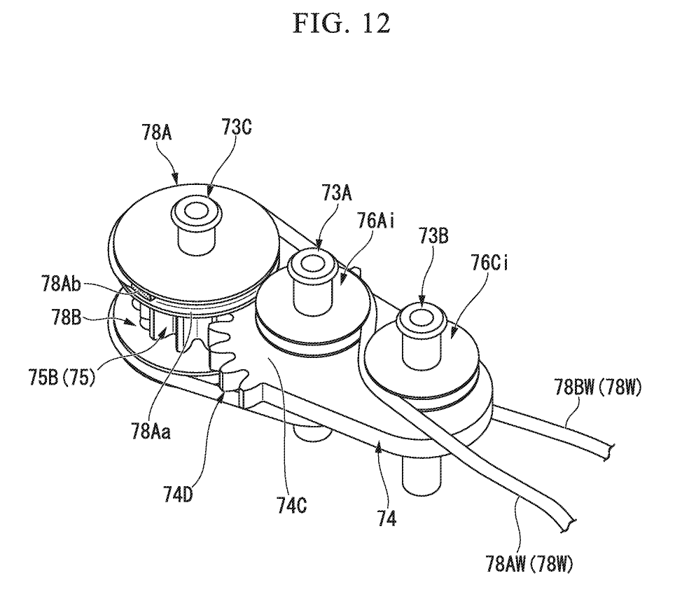

Subsequently, the intermediate joint 70 will be described with reference to FIG. 10 to FIG. 14. FIG. 10 is a part of a cross-sectional view illustrating a configuration of the intermediate joint 70. FIG. 11 is an exploded perspective view of the intermediate joint 70. FIG. 12 is a perspective view illustrating a configuration of an intermediate connection member 74 and a rotation-transmitting portion 75 of the intermediate joint 70.

The intermediate joint 70 is a double joint articulation bent around the direction Y1. The intermediate joint 70 has an axis (first axis, fourth axis) O70A, an axis (second axis, fifth axis) O70B, and an axis (third axis, sixth axis) O70C. The axis O70B is provided to be separated from the axis O70A and is arranged to be parallel to the axis O70A. In addition, the axis O70B is arranged on the proximal end side of the axis O70A. The axis O70C is provided to be separated from the axis O70A and the axis O70B and is arranged to be parallel to the axis O70A and the axis O70B. In addition, the axis O70C is arranged on the distal end side of the axis O70A, and its position is fixed with respect to the axis O70A. In the present embodiment, the axis O70A, the axis O70B, and the axis O70C are orthogonal to the longitudinal axis O1 and are parallel to the direction Y1.

In addition, the intermediate joint 70 has a distal end connection member (first connection member, fourth connection member) 71, a proximal end connection member (second connection member, fifth connection member) 72, an intermediate connection member (third connection member, sixth connection member) 74, and a rotation-transmitting portion (third rotation-transmitting portion, seventh rotation-transmitting portion) 75. In the distal end joint 30, the rotation-transmitting portion 35 is arranged on the proximal end side of the intermediate connection member 34. In contrast, in the intermediate joint 70, the rotation-transmitting portion 75 is arranged on the distal end side of the intermediate connection member 74.

The distal end connection member 71 is fixed to the proximal end side of a proximal end portion 62 of the arm 60. The distal end connection member 71 has a rotation-transmitting portion (first rotation-transmitting portion, fifth rotation-transmitting portion) 71A provided on the proximal end side. The rotation-transmitting portion 71A is configured to be pivotable about the axis O70A. A circular hole 71B penetrating the rotation-transmitting portion 71A in the direction Y1 about the axis O70A is formed in the rotation-transmitting portion 71A. A pivot axis 73A formed into a columnar shape extending in the direction Y1 and having the axis O70A as its center axis is inserted through the circular hole 71B. The pivot axis 73A is relatively and pivotably supported by the rotation-transmitting portion 71A via the circular hole 71B about the axis O70A. That is, the rotation-transmitting portion 71A is pivotable about the axis O70A with respect to the pivot axis 73A.

The rotation-transmitting portion 71A is divided into an upper rotation-transmitting portion (first part, fifth part) 71Aa and a lower rotation-transmitting portion (second part, sixth part) 71Ab in a direction along the axis O70A, that is, in the direction Y1 (or the direction Y2). The upper rotation-transmitting portion 71Aa and the lower rotation-transmitting portion 71Ab are separated from each other in the direction Y1 and are arranged to be parallel to each other with the longitudinal axis O1 interposed therebetween. The upper rotation-transmitting portion 71Aa is arranged on the direction Y1 side of the longitudinal axis O1, and the lower rotation-transmitting portion 71Ab is arranged on the direction Y2 side of the longitudinal axis O1. In addition, as the circular hole 71B, a circular hole 71B a penetrating the upper rotation-transmitting portion 71Aa in the direction Y1 about the axis O70A is formed in the upper rotation-transmitting portion 71Aa, and a circular hole 71Bb penetrating the lower rotation-transmitting portion 71Ab in the direction Y1 about the axis O70A is formed in the lower rotation-transmitting portion 71Ab. The pivot axis 73A is inserted through the circular hole 71Ba and the circular hole 71Bb.

The rotation-transmitting portion 71A includes a gear portion (first gear portion, fifth gear portion) 71C which has a sector-shaped site (first part, fourth site) 71Cs about the axis O70A and in which teeth are formed on an arc of a sector-shaped site 71Cs. As the gear portion 71C, the upper rotation-transmitting portion 71Aa has an upper gear portion (first gear portion, fifth gear portion) 71Ca, and the lower rotation-transmitting portion 71Ab has a lower gear portion (first gear portion, fifth gear portion) 71Cb. As the sector-shaped site 71Cs, the upper gear portion 71Ca has a sector-shaped site (first part, fourth site) 71Cas, and the lower rotation-transmitting portion 71Ab has a sector-shaped site (first part, fourth site) 71Cbs. The gear portion 71C is configured to engage with a gear portion (second gear portion, sixth gear portion) 72C of a rotation-transmitting portion 72A (which will be described below). Specifically, the upper gear portion 71Ca engages with an upper gear portion 72Ca of the gear portion 72C, and the lower gear portion 71Cb engages with a lower gear portion 72Cb of the gear portion 72C. Consequently, the rotation-transmitting portion 71A and the rotation-transmitting portion 72A are connected to each other to pivot in an interlocked with each other.

The proximal end connection member 72 is provided on the distal end side of the arm 80 and is fixed to the arm 80. In the present embodiment, the proximal end connection member 72 is fixed to a distal end portion 91 of the proximal end joint 90 of the arm 80. The proximal end connection member 72 has the rotation-transmitting portion (second rotation-transmitting portion, sixth rotation-transmitting portion) 72A provided on the distal end side. The rotation-transmitting portion 72A is configured to be pivotable about the axis O70B. Specifically, a circular hole 72B penetrating the rotation-transmitting portion 72A in the direction Y1 about the axis O70B is formed in the rotation-transmitting portion 72A. A pivot axis 73B formed into a columnar shape extending in the direction Y1 and having the axis O70B as its center axis is inserted through the circular hole 72B. The pivot axis 73B is relatively and pivotably supported by the rotation-transmitting portion 72A via the circular hole 72Babout the axis O70B. That is, the rotation-transmitting portion 72A is pivotable about the axis O70B with respect to the pivot axis 73B.

The rotation-transmitting portion 72A is divided into an upper rotation-transmitting portion (third part, seventh part) 72Aa and a lower rotation-transmitting portion (fourth part, eighth part) 72Ab in a direction along the axis O70B, that is, in the direction Y1 (or the direction Y2). The upper rotation-transmitting portion 72Aa and the lower rotation-transmitting portion 72Ab are arranged in a form similar to that of the upper rotation-transmitting portion 71Aa and the lower rotation-transmitting portion 71Ab of the rotation-transmitting portion 71A. In addition, as the circular hole 72B, a circular hole 72Ba is formed in the upper rotation-transmitting portion 72Aa, and a circular hole 72Bb is formed in the lower rotation-transmitting portion 72Ab.

The rotation-transmitting portion 72A includes the gear portion (second gear portion, sixth gear portion) 72C which has a sector-shaped site (second part, fifth site) 72Cs about the axis O70B and in which teeth are formed on an arc of the sector-shaped site 72Cs. As the gear portion 72C, the upper rotation-transmitting portion 72Aa has an upper gear portion (second gear portion, sixth gear portion) 72Ca, and the lower rotation-transmitting portion 72Ab has a lower gear portion (second gear portion, sixth gear portion) 72Cb. As the sector-shaped site 72Cs, the upper gear portion 72Ca has a sector-shaped site (second part, fifth site) 72Cas, and the lower gear portion 72Cb has a sector-shaped site (second part, fifth site) 72Cbs.

In the present embodiment, the pitch circle diameter of the gear portion 71C (upper gear portion 71Ca and lower gear portion 71Cb) is the same as the pitch circle diameter of the gear portion 72C (upper gear portion 72Ca and lower gear portion 72Cb). In addition, the dimensions of the gear portion 71C in the direction Y1 are the same as the dimensions of the gear portion 72C in the direction Y1. That is, the dimensions of the upper gear portion 71Ca in the direction Y1 and the dimensions of the upper gear portion 72Ca in the direction Y1 are equal to each other, and the dimensions of the lower gear portion 71Cb in the direction Y1 and the dimensions of the lower gear portion 72Cb in the direction Y1 are equal to each other.

The intermediate connection member 74 is configured to be pivotable about the axis O70A and to be pivotable about the axis O70B. In the present embodiment, the intermediate connection member 74 has a similar configuration to that of the intermediate connection member 34 of the distal end joint 30, excluding that a rotation-transmitting portion 74C is provided on the distal end side. The pivot axis 73A is inserted through a circular hole 74A formed on the distal end side of the intermediate connection member 74 and is relatively and pivotably supported about the axis O70A. In addition, the pivot axis 73B is inserted through a circular hole 74B formed on the proximal end side of the intermediate connection member 74 and is relatively and pivotably supported about the axis O70B.

In the present embodiment, the distal end side of the intermediate connection member 74 is arranged between the upper rotation-transmitting portion 71Aa and the lower rotation-transmitting portion 71Ab of the rotation-transmitting portion 71A. The proximal end side of the intermediate connection member 74 is arranged between the upper rotation-transmitting portion 72Aa and the lower rotation-transmitting portion 72Ab of the rotation-transmitting portion 72A.

The intermediate connection member 74 has a rotation-transmitting portion (fourth rotation-transmitting portion, eighth rotation-transmitting portion) 74C provided in the distal end portion (end portion) of the intermediate connection member 74. The rotation-transmitting portion 74C is connected to the rotation-transmitting portion 75 to pivot about the axis O70A by interlocking with the rotation-transmitting portion 75. In the present embodiment, the rotation-transmitting portion 74C includes a gear portion (third gear portion, seventh gear portion) 74D which has a sector-shaped site (third part, sixth site) 74Ds about the axis O70B and in which teeth are formed on an arc of the sector-shaped site 74Ds. The gear portion 74D is configured to engage with a gear portion (fourth gear portion, eighth gear portion) 75B of the rotation-transmitting portion 75 (which will be described below). Therefore, rotational motion of the rotation-transmitting portion 75 is transmitted to the rotation-transmitting portion 74C via the gear portion 75B and the gear portion 74D. Consequently, the rotation-transmitting portion 74C can pivot about the axis O70A by interlocking with the rotation-transmitting portion 75.

A pulley 76Ai, a pulley 76Aj, and a pulley 76Ak are provided on the direction Y1 side of the upper rotation-transmitting portion 71Aa of the rotation-transmitting portion 71A along the direction Y1 in this order. A pulley 76Bi, a pulley 76Bj, and a pulley 76Bk are provided on the direction Y2 side of the lower rotation-transmitting portion 71Ab of the rotation-transmitting portion 71A along the direction Y2 in this order. A pulley 76Ci, a pulley 76Cj, and a pulley 76Ck are provided on the direction Y1 side of the upper rotation-transmitting portion 72Aa of the rotation-transmitting portion 72A along the direction Y1 in this order. A pulley 76Di, a pulley 76Dj, and a pulley 76Dk are provided on the direction Y2 side of the lower rotation-transmitting portion 72Ab of the rotation-transmitting portion 72A along the direction Y2 in this order. As the pulley 76Ai, the pulley 76Aj, the pulley 76Ak, the pulley 76Bi, the pulley 76Bj, the pulley 76Bk, the pulley 76Ci, the pulley 76Cj, the pulley 76Ck, the pulley 76Di, the pulley 76Dj, and the pulley 76Dk, it is possible to use pulleys having a similar configuration to that of the pulley 36A, the pulley 36B, the pulley 36C, or the pulley 36D of the distal end joint 30.

The pivot axis 73A is relatively and pivotably inserted through the circular hole of the pulley 76Ai, the circular hole of the pulley 76Aj, the circular hole of the pulley 76Ak, the circular hole of the pulley 76Bi, the circular hole of the pulley 76Bj, and the circular hole of the pulley 76Bk around the axis O70A. The pivot axis 73B is relatively and pivotably inserted through the circular hole of the pulley 76Ci, the circular hole of the pulley 76Cj, the circular hole of the pulley 76Ck, the circular hole of the pulley 76Di, the circular hole of the pulley 76Dj, and the circular hole of the pulley 76Dk around the axis O70B.

An operation wire 78AW extending from a pulley 78A (which will be described below) is wound around the pulley groove of the pulley 76Ai and the pulley groove of the pulley 76Ci. The operation wire 38AW extending from the pulley 38A of the distal end joint 30 is wound around the pulley groove of the pulley 76Aj and the pulley groove of the pulley 76Cj. The operation wire 25W extending from the pulley 36A of the distal end joint 30 is wound around the pulley groove of the pulley 76Ak and the pulley groove of the pulley 76Ck.

An operation wire 78BW extending from a pulley 78B (which will be described below) is wound around the pulley groove of the pulley 76Bi and the pulley groove of the pulley 76Di. The operation wire 38BW extending from the pulley 38B of the distal end joint 30 is wound around the pulley groove of the pulley 76Bj and the pulley groove of the pulley 76Dj. The operation wire 26W extending from the pulley 36B of the distal end joint 30 is wound around the pulley groove of the pulley 76Bk and the pulley groove of the pulley 76Dk.

In the present embodiment, as shown in FIG. 10, the pulley 76Ai and the pulley 76Ci, the pulley 76Aj and the pulley 76Cj, the pulley 76Ak and the pulley 76Ck, the pulley 76Bi and the pulley 76Di, the pulley 76Bj and the pulley 76Dj, and the pulley 76Bk and the pulley 76Dk are arranged at the same positions in the direction Y1.

In addition, the pulley 76Aj and the pulley 76Cj are arranged at the same positions as the pulley 38A in the direction Y1. The pulley 76Ak and the pulley 76Ck are arranged at the same positions as the pulley 36A and the pulley 36C in the direction Y1. The pulley 76Bj and the pulley 76Dj are arranged at the same positions as the pulley 38B in the direction Y1. The pulley 76Bk and the pulley 76Dk are arranged at the same positions as the pulley 36B and the pulley 36D in the direction Y1. Due to the configuration described above, it is possible to more smoothly drive the operation wires 25W, 26W, 38AW, and 38BW which straddle from the distal end joint 30 to the intermediate joint 70.

In addition, the operation wire 78AW is wound around from the direction X2 side of the pulley 76Ai to the direction X1 side of the pulley 76Ci in a manner intersecting the longitudinal axis O1 between the pulley 76Ai and the pulley 76Ci when seen in the direction Y2. The operation wire 38AW is wound around from the direction X1 side of the pulley 76Aj to the direction X2 side of the pulley 76Cj in a manner intersecting the longitudinal axis O1 between the pulley 76Aj and the pulley 76Cj when seen in the direction Y2. Similar to the operation wire 78AW wound around the pulley 76Ai and the pulley 76Ci, the operation wire 25W is wound around the pulley 76Ak and the pulley 76Ck. Similar to the operation wire 38AW wound around the pulley 76Aj and the pulley 76Cj, the operation wire 78BW is wound around the pulley 76Bi and the pulley 76Di. Similar to the operation wire 38BW wound around the pulley 76Ai and the pulley 76Ci, the operation wire 78AW is wound around the pulley 76Bj and the pulley 76Dj. Similar to the operation wire 38AW wound around the pulley 76Aj and the pulley 76Cj, the operation wire 26W is wound around the pulley 76Bk and the pulley 76Dk.

A support member 77A is provided on the direction Y1 side of the pulley 76Ak and the pulley 76Ck. The support member 77A can have a similar configuration to that of the support member 37A of the distal end joint 30. In addition, a support member 77B is provided on the direction Y2 side of the pulley 76Bk and the pulley 76Dk. The support member 77B can have a similar configuration to that of the support member 37B of the distal end joint 30.

The pivot axis 73A is relatively and pivotably inserted through a circular hole 77Aa of the support member 77A and a circular hole 77Ba of the support member 77B about the axis O70A. The pivot axis 73B is relatively and pivotably inserted through a circular hole 77Ab of the support member 77A and a circular hole 77Bb of the support member 77B about the axis O70B.

Both ends of the pivot axis 73A are respectively supported by the support member 77A and the support member 77B via the circular hole 77Aa and the circular hole 77Ba. Similarly, both ends of the pivot axis 73B are respectively supported by the support member 77A and the support member 77B via the circular hole 77Ab and the circular hole 77Bb.

Due to the configuration described above, the pivot axis 73A relatively and pivotably connects the support member 77A, the pulley 76Ai, the pulley 76Aj, the pulley 76Ak, the rotation-transmitting portion 71A (upper rotation-transmitting portion 71Aa and lower rotation-transmitting portion 71Ab), the intermediate connection member 74, the pulley 76Bi, the pulley 76Bj, the pulley 76Bk, and the support member 77B to each other about the axis O70A. Similarly, the pivot axis 73B relatively and pivotably connects the support member 77A, the pulley 76Ci, the pulley 76Cj, the pulley 76Ck, the rotation-transmitting portion 72A (upper rotation-transmitting portion 72Aa and lower rotation-transmitting portion 72Ab), the intermediate connection member 74, the pulley 76Di, the pulley 76Dj, the pulley 76Dk, and the support member 77B to each other about the axis O70B. In addition, the distance between the axis O70A and the axis O70B is uniformly retained by the support member 77A, the intermediate connection member 74, and the support member 77B.

The rotation-transmitting portion 75 is configured to be pivotable about the axis O70C. Specifically, a circular hole 75A penetrating the rotation-transmitting portion 75 in the direction Y1 about the axis O70C is formed. A pivot axis 73C formed into a columnar shape extending in the direction Y1 and having the axis O70C as its center axis is inserted through the circular hole 75A. The pivot axis 73C is relatively and pivotably supported by the rotation-transmitting portion 75 via the circular hole 75A about the axis O70C. That is, the rotation-transmitting portion 75 is pivotable about the axis O70C with respect to the pivot axis 73C.

The rotation-transmitting portion 75 has the gear portion (fourth gear portion, eighth gear portion) 75B which is formed into a columnar shape about the axis O70C and in which teeth are formed on the outer circumferential surface around the axis O70C. As described above, the gear portion 75B engages with the gear portion 74D. In addition, the pitch circle diameter of the gear portion 75B is smaller than the pitch circle diameter of the gear portion 74D. Therefore, the rotational frequency of the gear portion 74D can be lower than the rotational frequency of the gear portion 75B, and the torque of the gear portion 74D can be higher than the torque of the gear portion 75B.

In addition, the intermediate joint 70 has a pulley (rotor) 78 which is pivotable about the axis O70C and an operation wire (operation member) 78W which is wound around the pulley 78. The pulley 78 is connected to the gear portion 75B to integrally pivot with the gear portion 75B. In the present embodiment, as the pulley 78, a pulley (rotor) 78A and a pulley (rotor) 78B are provided. As the operation wire 78W, an operation wire (operation member) 78AW and an operation wire (operation member) 78BW are provided.

As the pulley 78A and the pulley 78B, it is possible to use pulleys having a similar configuration to that of the pulley 38A or the pulley 38B of the distal end joint 30. The pivot axis 73C is relatively and pivotably inserted through the circular hole (not shown) of the pulley 78A and the circular hole (not shown) of the pulley 78B around the axis O70C. The pulley 78A is provided to be adjacent to the gear portion 75B in the direction Y1 and is fixed to the gear portion 75B to pivot together with the gear portion 75B. The pulley 78A is arranged at the same position as the pulley 76Ai and the pulley 76Ci in the direction Y1. In addition, the pulley 78B is provided to be adjacent to the gear portion 75B in the direction Y2 and is fixed to the gear portion 75B to pivot together with the gear portion 75B. The pulley 78B is arranged at the same position as the pulley 76Bi and the pulley 76Di in the direction Y1.

The operation wire 78AW is wound around a pulley groove 78Aa of the pulley 78A. One end of the operation wire 78AW is fixed to the pulley groove 78Aa by a fixing portion 78Ab. The other end of the operation wire 78AW is connected to a power-transmitting portion of the medical instrument 1. The operation wire 78AW is wound counterclockwise around the pulley groove 78Aa when seen in the direction Y2 from the other end toward one end of the operation wire 78AW. In addition, the operation wire 78BW is wound around a pulley groove 78Ba of the pulley 78B. One end of the operation wire 78BW is fixed to the pulley groove 78Ba by a fixing portion (not shown). The other end of the operation wire 78BW is connected to a power-transmitting portion of the medical instrument 1. The operation wire 78BW is wound clockwise around the pulley groove 78Ba when seen in the direction Y2 from the other end toward one end of the operation wire 78BW.

Due to the configuration described above, when the operation wire 78AW is pulled to the proximal end side, the gear portion 75B pivots clockwise together with the pulley 78A when seen in the direction Y2. In addition, when the operation wire 78BW is pulled to the proximal end side, the gear portion 75B pivots counterclockwise together with the pulley 78B when seen in the direction Y2.

In the present embodiment, the outer diameter of the pulley 78A and the pulley 78B is larger than the pitch circle diameter of the gear portion 75B. Accordingly, it is possible to reduce a tensile force of the operation wire 78AW and the operation wire 78BW required for the gear portion 75B to pivot.

An accommodation space 62A extending along the longitudinal axis O1 is formed inside the proximal end portion 62 of the arm 60. The accommodation space 62A is arranged on the distal end side of the distal end connection member 71 fixed to the proximal end portion 62. The accommodation space 62A communicates with a space formed between the upper rotation-transmitting portion 71Aa and the lower rotation-transmitting portion 71Ab of the rotation-transmitting portion 71A via an opening 62Aa in a direction along the longitudinal axis O1. In addition, a circular hole 62Ba and a circular hole 62Bb penetrating the proximal end portion 62 about the axis O70C are formed in the proximal end portion 62. The circular hole 62Ba and the circular hole 62Bb individually communicate with the accommodation space 62A and are arranged with the accommodation space 62A interposed therebetween.

The rotation-transmitting portion 75, the pulley 78A, and the pulley 78B are arranged on the accommodation space 62A. The gear portion 74D of the rotation-transmitting portion 74C passes through the opening 62Aa, is arranged inside the accommodation space 62A, and engages with the gear portion 75B of the rotation-transmitting portion 75. Both ends of the pivot axis 73C are relatively and pivotably supported by the circular hole 62Ba and the circular hole 62Bb about the axis O70C. The pivot axis 73C is supported by the proximal end portion 62 through the circular hole 62Ba and the circular hole 62Bb, and the pivot axis 73A is supported by the rotation-transmitting portion 71A of the distal end connection member 71 fixed to the proximal end portion 62. Therefore, the position of the axis O70C is fixed with respect to the axis O70A.

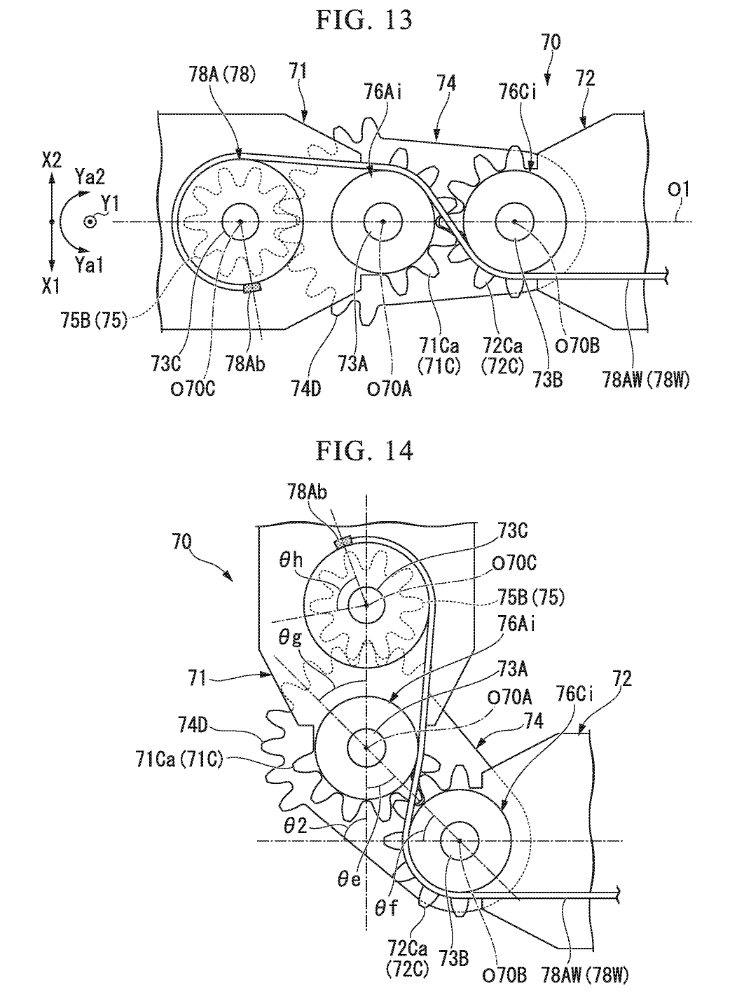

Movements of the intermediate joint 70 having the configuration described above will be described with reference to FIG. 13 and FIG. 14. FIG. 13 is a diagram illustrating a state where the intermediate joint 70 is straight. FIG. 14 is a diagram illustrating a state where the intermediate joint 70 is bent. FIG. 13 and FIG. 14 are diagrams of the intermediate joint 70 seen in the direction Y2. In FIG. 13 and FIG. 14, the configuration of the intermediate joint 70 is shown in a simplified manner.

As shown in FIG. 13, when the operation wire 78AW is pulled to the proximal end side in a state where the intermediate joint 70 is straight, the gear portion 75B fixed to the pulley 78A pivots clockwise, that is, about the axis O70C in the direction Ya2 when seen in the direction Y2. Since the gear portion 75B engages with the gear portion 74D of the intermediate connection member 74, the gear portion 74D pivots about the axis O70A in the direction Ya1 incidental to pivoting of the gear portion 75B. That is, the intermediate connection member 74 pivots about the axis O70A in the direction Ya1 with respect to the distal end connection member 71. In this case, the pivot axis 73B, that is, the axis O70B also rotatively moves about the axis O70A in the direction Ya1 together with the intermediate connection member 74. In addition, since the gear portion 72C of the proximal end connection member 72 engages with the gear portion 71C of the distal end connection member 71, the gear portion 72C pivots about the axis O70B in the direction Ya1 incidental to pivoting of the intermediate connection member 74. That is, the proximal end connection member 72 pivots about the axis O70B in the direction Ya1 with respect to the intermediate connection member 74. Due to the movement described above, as shown in FIG. 14, the intermediate joint 70 is in a state of being bent in the direction Ya1 with respect to the distal end connection member 71, in other words, is in a state of being bent in the direction Ya2 with respect to the proximal end connection member 72. In a case where the intermediate joint 70 is to be in a straight state from this state, or in a case where the intermediate joint 70 is to be bent in the direction Ya1 from a straight state, the operation wire 78BW is pulled to the proximal end side. Accordingly, the gear portion 75B fixed to the pulley 78B pivots about the axis O70C in the direction Ya1 opposite to the movement described above. Then, each of the members makes a movement in a direction opposite to that of the movement described above, so that the intermediate joint 70 is bent in the direction Ya1.

Bending movement of the above-described intermediate joint 70 will be described in more detail. Here, the radius of a pitch circle of the gear portion 71C is Re, the radius of a pitch circle of the gear portion 72C is Rf, the radius of a pitch circle of the gear portion 74D is Rg, and the radius of a pitch circle of the gear portion 75B is Rh. When the gear portion 75B pivots about the axis O70C in the direction Ya2 by an angle θh, in a case where the gear portion 74D pivots about the axis O70A in the direction Ya1 with respect to the distal end connection member 71 by an angle θg, the angle θg is expressed by the following expression.

θg=θh×Rh/Rg (6)

In this case, when an angle at which the gear portion 71C pivots about the axis O70A in the direction Ya2 with respect to the intermediate connection member 74 is θe and an angle at which the gear portion 72C pivots about the axis O70B in the direction Ya1 with respect to the intermediate connection member 74 is θf, the angle θe is expressed by the following expression.

θf=θe×Re/Rf (7)

In the present embodiment, the pitch circle diameter of the gear portion 71C and the pitch circle diameter of the gear portion 72C are equal to each other. In addition, the size of the angle θe and the size of the angle θg are the same as each other. Thus, Expression (7) is established as follows.

θf=θe=θg (8)

Therefore, an angle at which the proximal end connection member 72 pivots with respect to the distal end connection member 71, in other words, an angle θ2 at which the distal end connection member 71 pivots with respect to the proximal end connection member 72 is expressed by the following expression.

θ2=θg+θf=2θg (9)

That is, the angle θ2 at which the distal end connection member 71 pivots with respect to the proximal end connection member 72 becomes twice the angle θg at which the intermediate connection member 74 pivots with respect to the distal end connection member 71.

In addition, when the torque while the gear portion 74D pivots is Tg and the torque while the gear portion 75B pivots is Th, the torque Tg is expressed by the following expression.

Tg=Th×Rg/Rh (10)

In the present embodiment, since the pitch circle diameter of the gear portion 75B is smaller than the pitch circle diameter of the gear portion 74D, the relationship of Rg/Rh (reduction ratio)>1 is established. The torque Th of the gear portion 75B can be reduced with respect to the torque Tg of the gear portion 74D by increasing the reduction ratio in this manner. Thus, it is possible to reduce a tensile force applied to the operation wire 78AW and the operation wire 78BW when the gear portion 75B pivots.

In the intermediate joint 70 as well, similar to the distal end joint 30, bending of the articulation does not affect a path length of the operation wire.

In addition, in the intermediate joint 70, the rotation-transmitting portion 71A (upper rotation-transmitting portion 71Aa and lower rotation-transmitting portion 71Ab) of the distal end connection member 71, the rotation-transmitting portion 72A (upper rotation-transmitting portion 72Aa and lower rotation-transmitting portion 72Ab) of the proximal end connection member 72, and the intermediate connection member 74 are arranged similarly to the configuration of the distal end joint 30. Thus, similar to the distal end joint 30, the intermediate joint 70 is resistant to an external disturbance in a case where an external force of tilting the axis of the articulation acts.

The proximal end joint 90 is a double joint articulation bent around the direction Y1. Since the articulation structure of the proximal end joint 90 is similar to an articulation structure in which the distal end joint 30 is rotated around the longitudinal axis O1 by 90 degrees, detailed description thereof will not be repeated. In the distal end joint 30, two pulleys are respectively provided in the pivot axes. However, in the proximal end joint 90, six pulleys are respectively provided in the pivot axes to guide the operation wires 25W, 26W, 38AW, 38BW, 78AW, and 78BW. The proximal end side of the proximal end joint 90 is connected to the main body 50.

The main body 50 has a flexibly elongated portion 51 which is formed into a cylindrical shape extending along the longitudinal axis O1 (refer to FIG. 2) and a power-transmitting portion (not shown) which is provided in the proximal end of the elongated portion 51. The operation wires 25W, 26W, 38AW, 38BW, 78AW, and 78BW and the operation wire for driving the proximal end joint 90 are inserted through the inside of the elongated portion 51 and are connected to the power-transmitting portion. The power-transmitting portion has a drive source which generates a drive force for driving each of the operation wires. When each of the operation wires is suitably driven by the power-transmitting portion, the distal end joint 30, the intermediate joint 70, and the proximal end joint 90 can be bent.

According to the medical instrument 1 of the present embodiment, since the distal end joint 30, the intermediate joint 70, and the proximal end joint 90 have the above-described configuration, the influence of an external disturbance applied to an articulation can be reduced, so that it is possible to configure a double joint articulation resistant to an external disturbance.

In the distal end joint 30, the axis O30C is arranged on the proximal end side of the axis O30B. Therefore, a different articulation can be provided adjacent to the distal end side of the distal end joint 30. In addition, in the intermediate joint 70, the axis O70C is arranged on the distal end side of the axis O70A. Therefore, a different articulation, for example, the proximal end joint 90 can be provided adjacent to the proximal end side of the intermediate joint 70. In the medical instrument 1 according to the present embodiment, the distance between the articulations can be shortened as described above. An articulation having a configuration of the intermediate joint 70 may be arranged at a position of the distal end joint 30, and an articulation having a configuration of the distal end joint 30 may be arranged at a position of the intermediate joint 70.

In the distal end joint 30, since the pitch circle diameter of the gear portion 35B is smaller than the pitch circle diameter of the gear portion 34D, it is possible to reduce a tensile force applied to the operation wire 38AW and the operation wire 38BW in order to cause the gear portion 34D to pivot. In addition, since the outer diameter of the pulley 38A and the pulley 38B is larger than the pitch circle diameter of the gear portion 35B, it is possible to further reduce a tensile force applied to the operation wire 38AW and the operation wire 38BW in order to cause the gear portion 34D to pivot. Accordingly, it is possible to reduce a force applied to the articulation on the proximal end side of the distal end joint 30, for example, the pivot axis 73A and the pivot axis 73B of the intermediate joint 70 via the pulleys 76Aj, 76Bj, 76Cj, and 76Dj.

The present embodiment has described that the medical instrument 1 is used by being inserted through the channel of the endoscope 110. However, an external guide tube may be attached to the endoscope 110 and the medical instrument 1 may be inserted through a channel of the guide tube. In addition, the present embodiment has described that the endoscope 110 is a flexible endoscope. However, the endoscope 110 may be a rigid endoscope.

The present embodiment has described an example in which the medical instrument 1 is gripping forceps and the end effector 10 has the pair of gripping pieces 11. However, the present embodiment is not limited thereto. The end effector 10 may have a different treatment tool such as a high-frequency knife.

The present embodiment has described that the pitch circle diameter of the gear portion 35B is smaller than the pitch circle diameter of the gear portion 34D in the distal end joint 30. However, the pitch circle diameter of the gear portion 35B may be equal to the pitch circle diameter of the gear portion 34D. That is, the gear portion 34D does not have to be reduced in speed. Similarly, in the description of the intermediate joint 70, the pitch circle diameter of the gear portion 75B is smaller than the pitch circle diameter of the gear portion 74D. However, the pitch circle diameter of the gear portion 75B may be equal to the pitch circle diameter of the gear portion 74D. That is, the gear portion 74D does not have to be reduced in speed.

In the present embodiment, in the distal end joint 30, the rotation-transmitting portion 31A and the rotation-transmitting portion 32A are connected to each other by the gear portion 31C and the gear portion 32C to pivot in an interlocked manner, and rotational motion is transmitted between the rotation-transmitting portion 31A and the rotation-transmitting portion 32A. However, the present embodiment is not limited thereto. Rotational motion may be transmitted by bringing two rollers in which teeth are not formed to come into frictional contact with each other, or rotational motion may be transmitted by using a wire and a pulley. In addition, other known methods of transmitting rotational motion may be used. The same applies to transmitting rotational motion between the rotation-transmitting portion 34C and the rotation-transmitting portion 35, and the intermediate joint 70.

The present embodiment has described that the elongated portion 51 of the main body 50 is flexible. However, the elongated portion 51 may be rigid.

Hereinabove, a preferable embodiment of the present invention has been described. However, the present invention is not limited to the embodiment. The configuration can be subjected to addition, omission, replacement, and other changes without departing from the gist of the present invention.

According to an embodiment of the present invention described above, it is possible to provide a medical instrument having a double joint articulation resistant to an external disturbance.

While preferred embodiments of the invention have been described and shown above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. A medical instrument, comprising:
a first joint which is provided between a first arm that is arranged in a distal end side of the medical instrument and a second arm that is arranged in a proximal end side of the medical instrument,
wherein the first joint has
a first axis which is a pivot center of the first joint,
a second axis which is provided to be separated from the first axis and is arranged to be parallel to the first axis,
a third axis which is provided to be separated from both of the first axis and the second axis and is arranged to be parallel to the first axis and the second axis,
a first connection member which has a first rotation-transmitting portion that is pivotable about the first axis and is fixed to the first arm,
a second connection member which has a second rotation-transmitting portion that is pivotable about the second axis and is fixed to the second arm,
a third connection member which is pivotable about the first axis and is pivotable about the second axis, and
a third rotation-transmitting portion which is pivotable about the third axis,
wherein the first rotation-transmitting portion and the second rotation-transmitting portion are connected to each other so as to pivot by interlocking with each other, and
wherein the third connection member has a fourth rotation-transmitting portion which is provided in an end portion of the third connection member and which is connected to the third rotation-transmitting portion so as to pivot about the first axis or the second axis by interlocking with the third rotation-transmitting portion.

2. The medical instrument according to claim 1,
wherein the third axis is arranged on a proximal end side of the second axis and a position of the third axis is fixed with respect to the second axis, and
wherein the fourth rotation-transmitting portion is connected to the third rotation-transmitting portion to pivot about the second axis in a manner being interlocked with the third rotation-transmitting portion.

3. The medical instrument according to claim 2,
wherein the first rotation-transmitting portion has a sector-shaped first site centering on the first axis and has a first gear portion in which teeth are formed on an arc of the first site,
wherein the second rotation-transmitting portion has a sector-shaped second site centering on the second axis and has a second gear portion in which teeth are formed on an arc of the second site,
wherein the first gear portion and the second gear portion engage with each other,
wherein the fourth rotation-transmitting portion has a sector-shaped third site centering on the second axis and has a third gear portion in which teeth are formed on an arc of the third site,
wherein the third rotation-transmitting portion has a fourth gear portion which is formed into a columnar shape centering on the third axis and in which teeth are formed on an outer circumferential surface around the third axis, and
wherein the third gear portion and the fourth gear portion engage with each other.

4. The medical instrument according to claim 3,
wherein a pitch circle diameter of the fourth gear portion is smaller than a pitch circle diameter of the third gear portion.

5. The medical instrument according to claim 3,
wherein the first joint further has
a rotor which is pivotable about the third axis, and
an operation member which is wound around the rotor, and
wherein the rotor is connected to the fourth gear portion to integrally pivot with the fourth gear portion.

6. The medical instrument according to claim 5,
wherein an outer diameter of the rotor is larger than the pitch circle diameter of the fourth gear portion.

7. The medical instrument according to claim 2,
wherein the first rotation-transmitting portion is divided into a first part and a second part in a direction along the first axis,
wherein the second rotation-transmitting portion is divided into a third part and a fourth part in a direction along the second axis, and
wherein the third connection member is arranged between the first part and the second part of the first rotation-transmitting portion and is arranged between the third part and the fourth part of the second rotation-transmitting portion.

8. The medical instrument according to claim 2,
wherein the second arm has
a third arm which is connected to the first joint,
a second joint which is connected to a proximal end side of the third arm, and
a fourth arm which connects a proximal end side of the second joint and the main body to each other,
wherein the second joint has
a fourth axis,
a fifth axis which is provided to be separated from the fourth axis and is arranged to be parallel to the fourth axis,
a sixth axis which is provided to be separated from both of the fourth axis and the fifth axis, which is arranged to be parallel to the fourth axis and the fifth axis on a distal end side of the fourth axis, and of which a position is fixed with respect to the fourth axis,
a fourth connection member which has a fifth rotation-transmitting portion pivotable about the fourth axis and is fixed to the third arm,
a fifth connection member which has a sixth rotation-transmitting portion pivotable about the fifth axis and is fixed to the fourth arm,
a sixth connection member which is pivotable about the fourth axis and is pivotable about the fifth axis, and
a seventh rotation-transmitting portion which is pivotable about the sixth axis, and
wherein the fifth rotation-transmitting portion and the sixth rotation-transmitting portion are connected to each other to pivot by interlocking with each other, and
wherein the sixth connection member has an eighth rotation-transmitting portion which is provided in an end portion of the sixth connection member and which is connected to the seventh rotation-transmitting portion to pivot about the fourth axis by interlocking with the seventh rotation-transmitting portion.

9. The medical instrument according to claim 8,
wherein the fifth rotation-transmitting portion has a sector-shaped fourth site centering on the fourth axis and has a fifth gear portion in which teeth are formed on an arc of the fourth site,
wherein the sixth rotation-transmitting portion has a sector-shaped fifth site centering on the fifth axis and has a sixth gear portion in which teeth are formed on an arc of the fifth site,
wherein the fifth gear portion and the sixth gear portion engage with each other,
wherein the eighth rotation-transmitting portion has a sector-shaped sixth site centering on the fourth axis and has a seventh gear portion in which teeth are formed on an arc of the sixth site,
wherein the seventh rotation-transmitting portion has an eighth gear portion which is formed into a columnar shape centering on the sixth axis and in which teeth are formed on an outer circumferential surface around the sixth axis, and
wherein the seventh gear portion and the eighth gear portion engage with each other.

10. The medical instrument according to claim 9,
wherein a pitch circle diameter of the eighth gear portion is smaller than a pitch circle diameter of the seventh gear portion.

11. The medical instrument according to claim 1,
wherein the third axis is arranged on a distal end side of the first axis and a position of the third axis is fixed with respect to the first axis, and
wherein the fourth rotation-transmitting portion is connected to the third rotation-transmitting portion to pivot about the first axis by interlocking with the third rotation-transmitting portion.

12. The medical instrument according to claim 11,
wherein the first rotation-transmitting portion has a sector-shaped first site centering on the first axis and has a first gear portion in which teeth are formed on an arc of the first site,
wherein the second rotation-transmitting portion has a sector-shaped second site centering on the second axis and has a second gear portion in which teeth are formed on an arc of the second site,
wherein the first gear portion and the second gear portion engage with each other,
wherein the fourth rotation-transmitting portion has a sector-shaped third site centering on the first axis and has a third gear portion in which teeth are formed on an arc of the third site,
wherein the third rotation-transmitting portion has a fourth gear portion which is formed into a columnar shape centering on the third axis and in which teeth are formed on an outer circumferential surface around the third axis, and
wherein the third gear portion and the fourth gear portion engage with each other.

13. The medical instrument according to claim 12,
wherein a pitch circle diameter of the fourth gear portion is smaller than a pitch circle diameter of the third gear portion.

14. The medical instrument according to claim 12,
wherein the first joint further has a rotor which is pivotable about the third axis, and
an operation member which is wound around the rotor, and
wherein the rotor is connected to the fourth gear portion to integrally pivot with the fourth gear portion.

15. The medical instrument according to claim 14,
wherein an outer diameter of the rotor is larger than the pitch circle diameter of the fourth gear portion.

16. The medical instrument according to claim 11,
wherein the first rotation-transmitting portion is divided into a first part and a second part in a direction along the first axis,
wherein the second rotation-transmitting portion is divided into a third part and a fourth part in a direction along the second axis, and
wherein the third connection member is arranged between the first part and the second part of the first rotation-transmitting portion and is arranged between the third part and the fourth part of the second rotation-transmitting portion.

17. A medical instrument, comprising:
a first rotation means for rotating a first connection member that is fixed to an arm;
a second rotation means for rotating a first connection member with respect to a second connection member, the second rotation means being provided in the second connection member that interlocks with the first connection member;
a third rotation means for rotating in the second connection member, the third rotation means being arranged opposed to the first rotation means with respect to the second rotation means;
a fourth rotation means for rotating around the first rotation means, the fourth rotation means being provided to a third connection member that is connected to the first connection member; and
an operation means for operating the third rotation means, wherein
the third connection member that interlocks with the third rotation means rotates with respect to the first rotation means by the fourth rotation means by operating the third rotation means by the operation means, and the first connection member is bent with respect to the second connection member by rotating the first connection member around the first rotation means by the second rotation means.

18. The medical instrument according to claim 17, wherein
a rotation direction of the first connection member is changed with respect to the second connection member by changing the rotation direction of the fourth rotation means with respect to the third rotation means by the operation means.
